(19) United States
(12) Patent Application Publication
(10) Pub. No.: US 2011/0064670 A1
(43) Pub. Date: Mar. 17, 2011

(54) METHOD TO IDENTIFY A PATIENT WITH AN INCREASED LIKELIHOOD OF RESPONDING TO AN ANTI-CANCER AGENT

(75) Inventors: Alvin Gogineni, San Jose, CA (US); Maresa Caunt, San Francisco, CA (US); Nicholas van Bruggen, San Carlos, CA (US); Anil D. Bagri, San Carlos, CA (US); Robby Weimer, Half Moon Bay, CA (US)

(73) Assignee: Genentech, Inc., South San Francisco, CA (US)

(21) Appl. No.: 12/879,234

(22) Filed: Sep. 10, 2010

Related U.S. Application Data
(60) Provisional application No. 61/241,769, filed on Sep. 11, 2009.

(51) Int. Cl. A61K 49/00; A61P 35/00; A61P 43/00

(52) U.S. Cl. 424/9.2

(57) ABSTRACT

The invention provides methods for identifying patients having an increased likelihood of responding to an anti-cancer agent or an increased likelihood of undergoing metastasis. The invention also provides methods for monitoring a patients' response to an anti-cancer agent. The invention also provides kits and articles of manufacture for use in the methods.

### RELATED APPLICATIONS

The present application claims the benefit of U.S. Provisional Patent Application No. 61/241,769, filed Sep. 11, 2009, the disclosure of which is hereby incorporated by reference in its entirety for all purposes.

### FIELD OF THE INVENTION

The present invention is directed to methods for identifying which patients will most benefit from treatment with anti-cancer-agents and monitoring patients for their sensitivity and responsiveness to treatment with anti-cancer agents.

### BACKGROUND OF THE INVENTION

Cancer is one of the most deadly threats to human health. In the U.S. alone, cancer affects nearly 1.3 million new patients each year, and is the second leading cause of death after cardiovascular disease, accounting for approximately 1 in 4 deaths. Solid tumors are responsible for most of those deaths. Although there have been significant advances in the medical treatment of certain cancers, the overall 5-year survival rate for all cancers has improved only by about 10% in the past 20 years. Cancers, or malignant tumors, metastasize and grow rapidly in an uncontrolled manner, making timely detection and treatment extremely difficult.

Depending on the cancer type, patients typically have several treatment options available to them including chemotherapy, radiation and antibody-based drugs. Diagnostic methods useful for predicting clinical outcome from the different treatment regimens would greatly benefit clinical management of these patients.

Thus, there is a need for more effective means for determining which patients will respond to which treatment and for incorporating such determinations into more effective treatment regimens for patients with anti-cancer therapies, whether used as single agents or combined with other agents.

### SUMMARY OF THE INVENTION

The present invention provides methods for identifying patients who will respond to treatment with anti-cancer agents.

One embodiment of the invention provide methods of identifying a patient likely to be responsive to an anti-cancer agent. The methods comprise (a) administering an imaging agent to a patient who has received at least one dose of an anti-cancer agent; (b) detecting lymph pulsation frequency in a lymph vessel associated with a tumor draining lymph node in the patient; and (c) comparing the lymph pulsation frequency to the pulsation frequency in the lymph vessel prior to treatment with the anti-cancer agent, wherein a decrease in lymph pulsation frequency in the lymph vessel of at least about 10% identifies a patient who has an increased likelihood of being responsive to an anti-cancer agent. In some embodiments, the lymph vessel connects the inguinal lymph node to the axial lymph node. In some embodiments, the imaging agent comprises a fluorescent dye (e.g., Alexafluor680). In some embodiments, the lymph pulsation frequency is detected using fluorescence microscopy. In some embodiments, the patient is a human. In some embodiments, the patient has been diagnosed with a cancer selected from colorectal cancer, breast cancer, lung cancer, glioblastoma, renal cancer, and combinations thereof. In some embodiments the methods further comprise (d) administering an effective amount of an anti-cancer agent to the patient if a decrease in lymph pulsation frequency in the lymph vessel of at least about 10% is detected. In some embodiments, the anti-cancer agent is selected from an NRP2 antagonist, a VEGF-C antagonist, and combinations thereof. In some embodiments, the NRP2 antagonist is an anti-NRP2 antibody. In some embodiments, the VEGF-C antagonist is an anti-VEGF-C antibody. In some embodiments, the methods further comprise (e) administering an effective amount of a second anti-cancer agent to the patient. In some embodiments, the second anti-cancer agent is a VEGF antagonist. In some embodiments, the VEGF antagonist is an anti-VEGF antibody. In some embodiments, the anti-VEGF antibody is bevacizumab.

Another embodiment of the invention provides methods of identifying a patient who has an increased likelihood of undergoing metastasis. The methods comprise (a) administering an imaging agent to a patient who has received at least one dose of an anti-cancer agent; (b) detecting lymph pulsation frequency in a lymph vessel associated with a tumor draining lymph node in the patient; and (c) comparing the lymph pulsation frequency to the pulsation frequency in the lymph vessel prior to treatment with the anti-cancer agent, wherein an increase in the lymph pulsation frequency in the lymph vessel of at least about 10% identifies a patient who has an increased likelihood of undergoing metastasis. In some embodiments, the lymph vessel connects the inguinal lymph node to the axial lymph node. In some embodiments, the imaging agent comprises a fluorescent dye. In some embodiments, the fluorescent dye is Alexafluor680. In some embodiments, lymph pulsation frequency is detected using fluorescence microscopy. In some embodiments, the patient is a human. In some embodiments, the patient has been diagnosed with a cancer selected from colorectal cancer, breast cancer, lung cancer, glioblastoma, renal cancer, and combinations thereof. In some embodiments, the methods further comprise (d) administering an effective amount of an anti-cancer agent to the patient if an increase in lymph pulsation frequency in the lymph vessel of at least about 10% is detected. In some embodiments, the anti-cancer agent is a member selected from the group consisting of: an NRP2 antagonist, a VEGF-C antagonist, and combinations thereof. In some embodiments, the NRP2 antagonist is an anti-NRP2 antibody. In some embodiments, the VEGF-C antagonist is an anti-VEGF-C antibody. In some embodiments, the methods further comprise (e) administering an effective amount of a second anti-cancer agent to the patient. In some embodiments, the second anti-cancer agent is a VEGF antagonist. In some embodiments, the VEGF antagonist is an anti-VEGF antibody. In some embodiments, the anti-VEGF antibody is bevacizumab.

A further embodiment of the invention provides methods for monitoring the effectiveness of anti-cancer therapy. The methods comprise (a) administering an imaging agent to a patient who has received at least one dose of an anti-cancer agent; (b) detecting lymph pulsation frequency in a lymph vessel associated with a tumor draining lymph node in the patient; and (c) comparing the lymph pulsation frequency to the pulsation frequency in the lymph vessel prior to treatment with the anti-cancer agent, wherein a decrease in lymph pulsation frequency in the lymph vessel of at least about 10% identifies an effective anti-cancer agent. In some embodiments, the lymph vessel connects the inguinal lymph node to the axial lymph node. In some embodiments, the imaging agent comprises a fluorescent dye. In some embodiments, the fluorescent dye is Alexafluor680. In some embodiments, lymph pulsation frequency is detected using fluorescence microscopy. In some embodiments, the patient is a human. In some embodiments, the patient has been diagnosed with a cancer selected from the group consisting of: colorectal cancer, breast cancer, lung cancer, glioblastoma, renal cancer, and combinations thereof. In some embodiments, the methods further comprise (d) administering an effective amount of an anti-cancer agent to the patient if a decrease in lymph pulsation frequency in the lymph vessel of at least about 10% is detected. In some embodiments, the anti-cancer agent is a member selected from the group consisting of: an NRP2 antagonist, a VEGF-C antagonist, and combinations thereof. In some embodiments, the NRP2 antagonist is an anti-NRP2 antibody. In some embodiments, the VEGF-C antagonist is an anti-VEGF-C antibody. In some embodiments, the methods further comprise (e) administering an effective amount of a second anti-cancer agent to the patient. In some embodiments, the second anti-cancer agent is a VEGF antagonist. In some embodiments, the VEGF antagonist is an anti-VEGF antibody. In some embodiments, the anti-VEGF antibody is bevacizumab.

Another embodiment of the invention provides methods of optimizing dose of an anti-cancer agent. The methods comprise (a) administering an imaging agent to a patient who has received at least one dose of an anti-cancer agent; (b) detecting lymph pulsation frequency in a lymph vessel associated with a tumor draining lymph node in the patient; and (c) comparing the lymph pulsation frequency to the pulsation frequency in the lymph vessel prior to treatment with the anti-cancer agent, wherein a change in lymph pulsation frequency in the lymph vessel identifies the dose as a minimum effective dose and no change in lymph pulsation frequency identifies the dose as a maximum effective dose. In some embodiments, the anti-cancer agent is selected from an NRP2 antagonist, a VEGF-C antagonist, and combinations thereof.

These and other embodiments are further described by the detailed description that follows.

### BRIEF DESCRIPTION OF THE DRAWINGS

FIG. 1 illustrates the results from a lymph function assay measuring lymph pulsation frequency. FIG. 1A illustrates a representative time course images of pulsatile lymph movement through a vessel following injection of a 15 μl bolus of dye. FIG. 1B illustrates a baseline activity of ˜24 events/5 min, n=6 animals.

FIG. 2 illustrates the results from a lymph function assay measuring bulk lymph transport to an inguinal lymph node following infusion of 5 μL/min, 15 min dye near base of the tail at the start of imaging. FIG. 2A illustrates representative time course images show initial loading of inguinal node followed by axial node. FIG. 2B illustrates baseline loading rate and time to maximum signal intensity of inguinal node, n=4 animals.

FIG. 3 illustrates the results from a lymph function assay demonstrating that bulk lymph transport is up-regulated in tumor associated lymph networks. FIG. 3A illustrates data demonstrating that lymph pulsation frequency is up-regulated ˜%50 in tumor implanted mice, n=6 animals /group. FIG. 3B illustrates data demonstrating that bulk lymph transport is also up-regulated in tumor implanted mice, n=4 animals/group. FIG. 3C illustrates data demonstrating the time course of lymph pulsation up-regulation in tumor implanted mice, n=12 animals/group.

FIG. 4 illustrates data demonstrating that inhibition of VEGF-C signaling decreases lymph transport in tumor associated networks. FIG. 4A illustrates data demonstrating that chronic treatment with anti-NRP2, anti-VEGF-C, or anti-VEGF-A in tumor-bearing mice significantly reduced lymph pulsation frequency, n=6 animals/group. FIG. 4B illustrates data demonstrating that chronic treatment with anti-NRP2, anti-VEGF-C, or anti-VEGF-A in tumor-bearing mice significantly reduced bulk lymph transport, n=6 animals/group.

FIG. 5 illustrates data demonstrating that inhibition of the VEGF-C pathway did not significantly alter lymphatic function in non-tumor bearing mice. FIG. 5A illustrates data demonstrating that chronic treatment with anti-VEGF-C in non-tumor-bearing mice did not significantly change lymph pulsation frequency when measured over 3 weeks, n=6 animals/group. FIG. 5B illustrates data demonstrating that chronic treatment with anti-NRP2 in non-tumor-bearing mice did not significantly change lymph pulsation frequency when measured over 3 weeks, n=4 animals/group.

FIG. 6 illustrates data demonstrating that acute injection of anti-cancer agents does not change lymphatic function. FIG. 6A illustrates data demonstrating that acute injection of anti-NRP2, anti-VEGF-C, or anti-VEGF-A in tumor bearing mice does not result in any significant change in lymph pulsation frequency, n=6 animals/group. FIG. 6B illustrates data demonstrating that acute injection of recombinant VEGF-C protein or recombinant VEGF-A protein in non-tumor bearing mice does not result in any significant change in lymph pulsation frequency, n=6 animals/group.

FIG. 7 illustrates data demonstrating that lymph pulsation frequency is up-regulated in both tail and back tumor bearing mice but is not up-regulated in the ear tumor bearing mice.

### DETAILED DESCRIPTION OF THE PREFERRED EMBODIMENTS

### I. Introduction

The invention provides methods for identifying patients having an increased likelihood of responding to an anti-cancer agent or an increased likelihood of undergoing metastasis. The invention also provides methods for monitoring a patients' response to an anti-cancer agent. The invention is based on the discovery that measurements of lymph function (e.g., pulsation frequency or bulk lymph transport) in tumor draining lymph vessels can be used to identify patients sensitive or responsive to treatment with anti-cancer agents or to identify patients with an increased likelihood of undergoing metastasis.

### II. Definitions

The term “lymph transport” refers to movement of lymph fluid through lymph vessels. Lymph vessels begin in tissues and carry or “drain” lymph fluid to local lymph nodes (e.g., cervical lymph nodes, axillary lymph nodes, supraclavicular lymph nodes, mediastinal lymph nodes, mesenteric lymph nodes, inguinal lymph nodes, and femoral lymph nodes) where the fluid is filtered and processed and sent to the next lymph node (e.g., cervical lymph nodes, axillary lymph nodes, supraclavicular lymph nodes, mediastinal lymph nodes, mesenteric lymph nodes, inguinal lymph nodes, and femoral lymph nodes) down the line until the fluid reaches the thoracic duct where it enters the blood stream. Any lymph node may be a draining lymph node. A “tumor draining lymph node” is any lymph node which receives lymph fluid from a tumor. Lymph transport includes, e.g., “lymph pulsation,” and “bulk lymph transport.” “Lymph pulsation” refers to lymph propulsion as it is pumped through lymph vessels.

In certain embodiments, the term “increase” refers to an overall increase of 5%, 10%, 20%, 25%, 30%, 40%, 50%, 60%, 70%, 80%, 85%, 90%, 95%, 96%, 97%, 98%, 99% or greater, in the lymph pulsation frequency in a lymph vessel detected by the methods described herein, as compared to the lymph pulsation frequency in the lymph vessel prior to treatment with an anti-cancer agent. In certain embodiments, the term increase refers to the increase in lymph pulsation frequency in a lymph vessel wherein the increase is at least about 1.5×, 1.75×, 2×, 3×, 4×, 5×, 6×, 7×, 8×, 9×, 10×, 25×, 50×, 75×, or 100× the lymph pulsation frequency in the lymph vessel prior to treatment with an anti-cancer agent.

In certain embodiments, the term “decrease” herein refers to an overall reduction of 5%, 10%, 20%, 25%, 30%, 40%, 50%, 60%, 70%, 80%, 85%, 90%, 95%, 96%, 97%, 98%, 99% or greater, in the lymph pulsation frequency in a lymph vessel detected by the methods described herein, as compared to the lymph pulsation frequency in the lymph vessel prior to treatment with an anti-cancer agent. In certain embodiments, the term decrease refers to the decrease in the lymph pulsation frequency in a lymph vessel detected by the methods described herein, as compared to a reference lymph vessel. wherein the decrease is at least about 0.9×, 0.8×, 0.7×, 0.6×, 0.5×, 0.4×, 0.3×, 0.2×, 0.1×, 0.05×, or 0.01× the lymph pulsation frequency in the lymph vessel prior to treatment with an anti-cancer agent.

“Imaging agent” refers to any compound that exhibits fluorescence at near-infrared wavelengths when exposed to excitation light. Examples of the imaging agents include, for example, indol-containing dyes, carbocyanine-containing dyes, polymethine dyes, acridines, anthraquinones, benzimidazols, indolenines, napthalimides, oxazines, oxonols, polyenes, porphins, squaraines, styryls, thiazols, xanthins, other NIR dyes known to those of skill in the art, or combinations thereof. The imaging agents typically have an excitation wavelength in the near-infrared range. In particular, the imaging agents may have excitation wavelengths of from about 550 nm to about 1000 nm, about 600 nm to about 950 nm, about 700 nm to about 900 nm, or about 750 nm to about 850 nm.

The terms “Neuropilin 2”, “NRP2” or “Nrp2” are used interchangeably and refer collectively to neuropilin-2 (NRP2, Nrp2) and its isoforms and variants, as described in Rossignol et al. (2000) Genomics 70:21 1-222. Neuropilins are 120 to 130 kDa non-tyrosine kinase receptors. There are multiple NRP-2 splice variants and soluble isoforms. The basic structure of neuropilins comprises five domains: three extracellular domains (ala2, blb2 and c), a transmembrane domain, and a cytoplasmic domain. The ala2 domain is homologous to complement components CIr and CIs (CUB), which generally contains four cysteine residues that form two disulfide bridges. The blb2 domain is homologous to coagulation factors V and VIII. The central portion of the c domain is designated as MAM due to its homology to meprin, A5 and receptor tyrosine phosphotase μ proteins. The ala2 and blb2 domains are responsible for ligand binding, whereas the c domain is critical for homodimerization or heterodimerization. Gu et al. (2002) J. Biol. Chem. 211; 18069-76; He and Tessier-Lavigne (1997) Cell 90:739-51.

“Neuropilin mediated biological activity” refers in general to physiological or pathological events in which neuropilin-1 and/or neuropilin-2 plays a substantial role. Non-limiting examples of such activities are axon guidance during embryonic nervous system development or neuron-regeneration, angiogenesis (including vascular modeling), tumorgenesis and tumor metastasis.

“Neuropilin-2 mediated biological activity” or “Nrp2 mediated biological activity,” as used herein, refers in general to physiological or pathological events in which Nrp2 plays a substantial role, such as, for example, enhancing VEGF receptor activation, and, in particular, the ability to modulate lymphatic endothelial cell (EC) migration, role in adult lymphangiogenesis, especially tumoral lymphangiogenesis and tumor metastasis.

The terms “vascular endothelial growth factor-C”, “VEGF-C”, “VEGFC”, “VEGF-related protein”, “VRP”, “VEGF2” and “VEGF-2” are used interchangeably, and refer to a member of the VEGF family, is known to bind at least two cell surface receptor families, the tyrosine kinase VEGF receptors and the neuropilin (Nrp) receptors. Of the three VEGF receptors, VEGF-C can bind VEGFR2 (KDR receptor) and VEGFR3 (Flt-4 receptor) leading to receptor dimerization (Shinkai et al., J Biol Chem 273, 31283-31288 (1998)), kinase activation and autophosphorylation (Heldin, Cell 80, 213-223 (1995); Waltenberger et al., J. Biol Chem 269, 26988-26995 (1994)). The phosphorylated receptor induces the activation of multiple substrates leading to angiogenesis and lymphangiogenesis (Ferrara et al., Nat Med 9, 669-676 (2003)). Overexpression of VEGF-C in tumor cells was shown to promote tumor-associated lymphangiogenesis, resulting in enhanced metastasis to regional lymph nodes (Karpanen et al., Faseb J 20, 1462-1472 (2001); Mandriota et al., EMBO J. 20, 672-682 (2001); Skobe et al., Nat Med 7, 192-198 (2001); Stacker et al., Nat Rev Cancer 2, 573-583 (2002); Stacker et al., Faseb J 16, 922-934 (2002)). VEGF-C expression has also been correlated with tumor-associated lymphangiogenesis and lymph node metastasis for a number of human cancers (reviewed in Achen et al., 2006, supra. In addition, blockade of VEGF-C-mediated signaling has been shown to suppress tumor lymphangiogenesis and lymph node metastases in mice (Chen et al., Cancer Res 65, 9004-9011 (2005); He et al., J. Natl Cancer Inst 94, 8190825 (2002); Krishnan et al., Cancer Res 63, 713-722 (2003); Lin et al., Cancer Res 65, 6901-6909 (2005)).

“Vascular endothelial growth factor-C”, “VEGF-C”, “VEGFC”, “VEGF-related protein”, “VRP”, “VEGF2” and “VEGF-2” refer to the full-length polypeptide and/or the active fragments of the full-length polypeptide. In one embodiment, active fragments include any portions of the full-length amino acid sequence which have less than the full 419 amino acids of the full-length amino acid sequence as shown in SEQ ID NO:3 of U.S. Pat. No. 6,451,764, the entire disclosure of which is expressly incorporated herein by reference. Such active fragments contain VEGF-C biological activity and include, but not limited to, mature VEGF-C. In one embodiment, the full-length VEGF-C polypeptide is proteolytically processed produce a mature form of VEGF-C polypeptide, also referred to as mature VEGF-C. Such processing includes cleavage of a signal peptide and cleavage of an amino-terminal peptide and cleavage of a carboxyl-terminal peptide to produce a fully-processed mature form. Experimental evidence demonstrates that the full-length VEGF-C, partially-processed forms of VEGF-C and fully processed mature forms of VEGF-C are able to bind VEGFR3 (Flt-4 receptor). However, high affinity binding to VEGFR2 occurs only with the fully processed mature forms of VEGF-C.

The term “biological activity” and “biologically active” with regard to a VEGF-C polypeptide refer to physical/chemical properties and biological functions associated with full-length and/or mature VEGF-C. In some embodiments, VEGF-C “biological activity” means having the ability to bind to, and stimulate the phosphorylation of, the Flt-4 receptor (VEGFR3). Generally, VEGF-C will bind to the extracellular domain of the Flt-4 receptor and thereby activate or inhibit the intracellular tyrosine kinase domain thereof. Consequently, binding of VEGF-C to the receptor may result in enhancement or inhibition of proliferation and/or differentiation and/or activation of cells having the Flt-4 receptor for the VEGF-C in vivo or in vitro. Binding of VEGF-C to the Flt-4 receptor can be determined using conventional techniques, including competitive binding methods, such as RIAs, ELISAs, and other competitive binding assays. Ligand/receptor complexes can be identified using such separation methods as filtration, centrifugation, flow cytometry (see, e.g., Lyman et al., Cell, 75:1157-1167 [1993]; Urdal et al., J. Biol. Chem., 263:2870-2877 [1988]; and Gearing et al., EMBO J., 8:3667-3676 [1989]), and the like. Results from binding studies can be analyzed using any conventional graphical representation of the binding data, such as Scatchard analysis (Scatchard, Ann. NY Acad. Sci., 51:660-672 [1949]; Goodwin et al., Cell, 73:447-456 [1993]), and the like. Since VEGF-C induces phosphorylation of the Flt-4 receptor, conventional tyrosine phosphorylation assays can also be used as an indication of the formation of a Flt-4 receptor/VEGF-C complex. In another embodiment, VEGF-C “biological activity” means having the ability to bind to KDR receptor (VEGFR2). vascular permeability, as well as the migration and proliferation of endothelial cells. In certain embodiments, binding of VEGF-C to the KDR receptor may result in enhancement or inhibition of vascular permeability as well as migration and/or proliferation and/or differentiation and/or activation of endothelial cells having the KDR receptor for the VEGF-C in vivo or in vitro.

The term “VEGF-C antagonist” is used herein to refer to a molecule capable of neutralizing, blocking, inhibiting, abrogating, reducing or interfering with VEGF-C activities. In certain embodiments, VEGF-C antagonist refers to a molecule capable of neutralizing, blocking, inhibiting, abrogating, reducing or interfering with the ability of VEGF-C to modulate angiogenesis, lymphatic endothelial cell (EC) migration, proliferation or adult lymphangiogenesis, especially tumoral lymphangiogenesis and tumor metastasis. VEGF-C antagonists include, without limitation, anti-VEGF-C antibodies and antigen-binding fragments thereof, receptor molecules and derivatives which bind specifically to VEGF-C thereby sequestering its binding to one or more receptors, anti-VEGF-C receptor antibodies and VEGF-C receptor antagonists such as small molecule inhibitors of the VEGFR2 and VEGFR3. The term “VEGF-C antagonist,” as used herein, specifically includes molecules, including antibodies, antibody fragments, other binding polypeptides, peptides, and non-peptide small molecules, that bind to VEGF-C and are capable of neutralizing, blocking, inhibiting, abrogating, reducing or interfering with VEGF-C activities. Thus, the term “VEGF-C activities” specifically includes VEGF-C mediated biological activities (as hereinabove defined) of VEGF-C.

The term “anti-VEGF-C antibody” or “an antibody that binds to VEGF-C” refers to an antibody that is capable of binding VEGF-C with sufficient affinity such that the antibody is useful as a diagnostic and/or therapeutic agent in targeting VEGF-C. Anti-VEGF-C antibodies are described, for example, in Attorney Docket PR4391, the entire content of the patent application is expressly incorporated herein by reference. In one embodiment, the extent of binding of an anti-VEGF-C antibody to an unrelated, non-VEGF-C protein is less than about 10% of the binding of the antibody to VEGF-C as measured, e.g., by a radioimmunoassay (RIA). In certain embodiments, an antibody that binds to VEGF-C has a dissociation constant (Kd) of ≦1 μM, ≦100 nM, ≦10 nM, ≦1 nM, or ≦0.1 nM. In certain embodiments, an anti-VEGF-C antibody binds to an epitope of VEGF-C that is conserved among VEGF-C from different species.

The term “VEGF” or “VEGF-A” as used herein refers to the 165-amino acid human vascular endothelial cell growth factor and related 121-, 189-, and 206-amino acid human vascular endothelial cell growth factors, as described by Leung et al. (1989) Science 246:1306, and Houck et al. (1991) Mol. Endocrin, 5:1806, together with the naturally occurring allelic and processed forms thereof. The term “VEGF” also refers to VEGFs from non-human species such as mouse, rat or primate. Sometimes the VEGF from a specific species are indicated by terms such as hVEGF for human VEGF, mVEGF for murine VEGF, and etc. The term “VEGF” is also used to refer to truncated forms of the polypeptide comprising amino acids 8 to 109 or 1 to 109 of the 165-amino acid human vascular endothelial cell growth factor. Reference to any such forms of VEGF may be identified in the present application, e.g., by “VEGF (8-109),” “VEGF (1-109)” or “VEGF165.” The amino acid positions for a “truncated” native VEGF are numbered as indicated in the native VEGF sequence. For example, amino acid position 17 (methionine) in truncated native VEGF is also position 17 (methionine) in native VEGF. The truncated native VEGF has binding affinity for the KDR and Flt-1 receptors comparable to native VEGF.

“VEGF biological activity” includes binding to any VEGF receptor or any VEGF signaling activity such as regulation of both normal and abnormal angiogenesis and vasculogenesis (Ferrara and Davis-Smyth (1997) Endocrine Rev. 18:4-25; Ferrara (1999) J. Mol. Med. 77:527-543); promoting embryonic vasculogenesis and angiogenesis (Carmeliet et al. (1996) Nature 380:435-439; Ferrara et al. (1996) Nature 380:439-442); and modulating the cyclical blood vessel proliferation in the female reproductive tract and for bone growth and cartilage formation (Ferrara et al. (1998) Nature Med. 4:336-340; Gerber et al. (1999) Nature Med. 5:623-628). In addition to being an angiogenic factor in angiogenesis and vasculogenesis, VEGF, as a pleiotropic growth factor, exhibits multiple biological effects in other physiological processes, such as endothelial cell survival, vessel permeability and vasodilation, monocyte chemotaxis and calcium influx (Ferrara and Davis-Smyth (1997), supra and Cebe-Suarez et al. Cell. Mol. Life. Sci. 63:601-615 (2006)). Moreover, recent studies have reported mitogenic effects of VEGF on a few non-endothelial cell types, such as retinal pigment epithelial cells, pancreatic duct cells, and Schwann cells. Guerrin et al. (1995) J. Cell Physiol. 164:385-394; Oberg-Welsh et al. (1997) Mol. Cell. Endocrinol. 126:125-132; Sondell et al. (1999) J. Neurosci. 19:5731-5740.

A “VEGF antagonist” or “VEGF-specific antagonist” refers to a molecule capable of binding to VEGF, reducing VEGF expression levels, or neutralizing, blocking, inhibiting, abrogating, reducing, or interfering with VEGF biological activities, including, but not limited to, VEGF binding to one or more VEGF receptors and VEGF mediated angiogenesis and endothelial cell survival or proliferation. Included as VEGF-specific antagonists useful in the methods of the invention are polypeptides that specifically bind to VEGF, anti-VEGF antibodies and antigen-binding fragments thereof, receptor molecules and derivatives which bind specifically to VEGF thereby sequestering its binding to one or more receptors, fusions proteins (e.g., VEGF-Trap (Regeneron)), and VEGF₁₂₁-gelonin (Peregrine). VEGF-specific antagonists also include antagonist variants of VEGF polypeptides, antisense nucleobase oligomers directed to VEGF, small RNA molecules directed to VEGF, RNA aptamers, peptibodies, and ribozymes against VEGF. VEGF-specific antagonists also include nonpeptide small molecules that bind to VEGF and are capable of blocking, inhibiting, abrogating, reducing, or interfering with VEGF biological activities. Thus, the term “VEGF activities” specifically includes VEGF mediated biological activities of VEGF. In certain embodiments, the VEGF antagonist reduces or inhibits, by at least 10%, 20%, 30%, 40%, 50%, 60%, 70%, 80%, 90% or more, the expression level or biological activity of VEGF.

An “anti-VEGF antibody” is an antibody that binds to VEGF with sufficient affinity and specificity. In certain embodiments, the antibody selected will normally have a sufficiently binding affinity for VEGF, for example, the antibody may bind hVEGF with a K_{d} value of between 100 nM-1 pM. Antibody affinities may be determined by a surface plasmon resonance based assay (such as the BIAcore assay as described in PCT Application Publication No. WO2005/012359); enzyme-linked immunoabsorbent assay (ELISA); and competition assays (e.g. RIA's), for example.

In certain embodiment, the anti-VEGF antibody can be used as a therapeutic agent in targeting and interfering with diseases or conditions wherein the VEGF activity is involved. Also, the antibody may be subjected to other biological activity assays, e.g., in order to evaluate its effectiveness as a therapeutic. Such assays are known in the art and depend on the target antigen and intended use for the antibody. Examples include the HUVEC inhibition assay; tumor cell growth inhibition assays (as described in WO 89/06692, for example); antibody-dependent cellular cytotoxicity (ADCC) and complement-mediated cytotoxicity (CDC) assays (U.S. Pat. No. 5,500,362); and agonistic activity or hematopoiesis assays (see WO 95/27062). An anti-VEGF antibody will usually not bind to other VEGF homologues such as VEGF-B or VEGF-C, nor other growth factors such as PlGF, PDGF or bFGF. In one embodiment, anti-VEGF antibody is a monoclonal antibody that binds to the same epitope as the monoclonal anti-VEGF antibody A4.6.1 produced by hybridoma ATCC HB 10709. In another embodiment, the anti-VEGF antibody is a recombinant humanized anti-VEGF monoclonal antibody generated according to Presta et al. (1997) Cancer Res. 57:4593-4599, including but not limited to the antibody known as bevacizumab (BV; AVASTIN®).

The anti-VEGF antibody “Bevacizumab (BV),” also known as “rhuMAb VEGF” or “AVASTIN®,” is a recombinant humanized anti-VEGF monoclonal antibody generated according to Presta et al. (1997) Cancer Res. 57:4593-4599. It comprises mutated human IgG1 framework regions and antigen-binding complementarity-determining regions from the murine anti-hVEGF monoclonal antibody A.4.6.1 that blocks binding of human VEGF to its receptors. Approximately 93% of the amino acid sequence of Bevacizumab, including most of the framework regions, is derived from human IgG1, and about 7% of the sequence is derived from the murine antibody A4.6.1. Bevacizumab has a molecular mass of about 149,000 daltons and is glycosylated. Bevacizumab and other humanized anti-VEGF antibodies are further described in U.S. Pat. No. 6,884,879 issued Feb. 26, 2005, the entire disclosure of which is expressly incorporated herein by reference.

The two best characterized VEGF receptors are VEGFR1 (also known as Flt-1) and VEGFR2 (also known as KDR and FLK-1 for the murine homolog). The specificity of each receptor for each VEGF family member varies but VEGF-A binds to both Flt-1 and KDR. The full length Flt-1 receptor includes an extracellular domain that has seven Ig domains, a transmembrane domain, and an intracellular domain with tyrosine kinase activity. The extracellular domain is involved in the binding of VEGF and the intracellular domain is involved in signal transduction.

VEGF receptor molecules, or fragments thereof, that specifically bind to VEGF can be used as VEGF inhibitors that bind to and sequester the VEGF protein, thereby preventing it from signaling. In certain embodiments, the VEGF receptor molecule, or VEGF binding fragment thereof, is a soluble form, such as sFlt-1. A soluble form of the receptor exerts an inhibitory effect on the biological activity of the VEGF protein by binding to VEGF, thereby preventing it from binding to its natural receptors present on the surface of target cells. Also included are VEGF receptor fusion proteins, examples of which are described below.

A chimeric VEGF receptor protein is a receptor molecule having amino acid sequences derived from at least two different proteins, at least one of which is a VEGF receptor protein (e.g., the flt-1 or KDR receptor), that is capable of binding to and inhibiting the biological activity of VEGF. In certain embodiments, the chimeric VEGF receptor proteins of the present invention consist of amino acid sequences derived from only two different VEGF receptor molecules; however, amino acid sequences comprising one, two, three, four, five, six, or all seven Ig-like domains from the extracellular ligand-binding region of the flt-1 and/or KDR receptor can be linked to amino acid sequences from other unrelated proteins, for example, immunoglobulin sequences. Other amino acid sequences to which Ig-like domains are combined will be readily apparent to those of ordinary skill in the art. Examples of chimeric VEGF receptor proteins include, but not limited to, soluble Flt-1/Fc, KDR/Fc, or Flt-1/KDR/Fc (also known as VEGF Trap). (See for example PCT Application Publication No. WO97/44453).

A soluble VEGF receptor protein or chimeric VEGF receptor proteins includes VEGF receptor proteins which are not fixed to the surface of cells via a transmembrane domain. As such, soluble forms of the VEGF receptor, including chimeric receptor proteins, while capable of binding to and inactivating VEGF, do not comprise a transmembrane domain and thus generally do not become associated with the cell membrane of cells in which the molecule is expressed.

Additional VEGF inhibitors are described in, for example in WO 99/24440, PCT International Application PCT/IB99/00797, in WO 95/21613, WO 99/61422, U.S. Pat. No. 6,534,524, U.S. Pat. No. 5,834,504, WO 98/50356, U.S. Pat. No. 5,883,113, U.S. Pat. No. 5,886,020, U.S. Pat. No. 5,792,783, U.S. Pat. No. 6,653,308, WO 99/10349, WO 97/32856, WO 97/22596, WO 98/54093, WO 98/02438, WO 99/16755, and WO 98/02437, all of which are herein incorporated by reference in their entirety.

The term “B20 series polypeptide” as used herein refers to a polypeptide, including an antibody that binds to VEGF. B20 series polypeptides includes, but not limited to, antibodies derived from a sequence of the B20 antibody or a B20-derived antibody described in US Publication No. 20060280747, US Publication No. 20070141065 and/or US Publication No. 20070020267, the content of these patent applications are expressly incorporated herein by reference. In one embodiment, B20 series polypeptide is B20-4.1 as described in US Publication No. 20060280747, US Publication No. 20070141065 and/or US Publication No. 20070020267. In another embodiment, B20 series polypeptide is B20-4.1.1 described in U.S. Patent Application 60/991,302, the entire disclosure of which is expressly incorporated herein by reference.

The term “G6 series polypeptide” as used herein refers to a polypeptide, including an antibody that binds to VEGF. G6 series polypeptides includes, but not limited to, antibodies derived from a sequence of the G6 antibody or a G6-derived antibody described in US Publication No. 20060280747, US Publication No. 20070141065 and/or US Publication No. 20070020267. G6 series polypeptides, as described in US Publication No. 20060280747, US Publication No. 20070141065 and/or US Publication No. 20070020267 include, but not limited to, G6-8, G6-23 and G6-31.

For additional antibodies see U.S. Pat. Nos. 7,060,269, 6,582,959, 6,703,020; 6,054,297; WO98/45332; WO 96/30046; WO94/10202; EP 0666868B1; U.S. Patent Application Publication Nos. 2006009360, 20050186208, 20030206899, 20030190317, 20030203409, and 20050112126; and Popkov et al., Journal of Immunological Methods 288:149-164 (2004). In certain embodiments, other antibodies include those that bind to a functional epitope on human VEGF comprising of residues F17, M18, D19, Y21, Y25, Q89, 191, K101, E103, and C104 or, alternatively, comprising residues F17, Y21, Q22, Y25, D63, I83 and Q89.

Other anti-VEGF antibodies are also known, and described, for example, in Liang et al., J Biol Chem 281, 951-961 (2006).

An “effective response” of a patient or a patient's “responsiveness” or “sensitivity” to treatment with an anti-cancer agent refers to the clinical or therapeutic benefit imparted to a patient at risk for or suffering from cancer from or as a result of the treatment with an anti-cancer agent, such as, e.g., an anti-VEGF-A antibody, an anti-VEGF-C antibody, or an anti-NRP2 antibody. Such benefit includes cellular or biological responses, a complete response, a partial response, a stable disease (without progression or relapse), or a response with a later relapse of the patient from or as a result of the treatment with the antagonist. For example, an effective response can be reduced tumor size or progression-free survival in a patient diagnosed as having a decrease in lymph pulsation frequency in a lymph vessel associated with a tumor draining lymph node following at least one treatment with an anti-cancer agent. The decrease in lymph pulsation frequency effectively predicts, or predicts with high sensitivity, such effective response.

“Antagonists as used herein refer to compounds or agents which inhibit or reduce the biological activity of the molecule to which they bind. Antagonists include antibodies, synthetic or native-sequence peptides, immunoadhesins, and small-molecule antagonists that bind to VEGF, optionally conjugated with or fused to another molecule. A “blocking” antibody or an “antagonist” antibody is one which inhibits or reduces biological activity of the antigen it binds.

An “agonist antibody,” as used herein, is an antibody which partially or fully mimics at least one of the functional activities of a polypeptide of interest.

The term “antibody” herein is used in the broadest sense and specifically covers monoclonal antibodies, polyclonal antibodies, multispecific antibodies (e.g. bispecific antibodies) formed from at least two intact antibodies, and antibody fragments so long as they exhibit the desired biological activity.

An “isolated” antibody is one which has been identified and separated and/or recovered from a component of its natural environment. Contaminant components of its natural environment are materials which would interfere with research, diagnostic or therapeutic uses for the antibody, and may include enzymes, hormones, and other proteinaceous or nonproteinaceous solutes. In some embodiments, an antibody is purified (1) to greater than 95% by weight of antibody as determined by, for example, the Lowry method, and in some embodiments, to greater than 99% by weight; (2) to a degree sufficient to obtain at least 15 residues of N-terminal or internal amino acid sequence by use of, for example, a spinning cup sequenator, or (3) to homogeneity by SDS-PAGE under reducing or nonreducing conditions using, for example, Coomassie blue or silver stain. Isolated antibody includes the antibody in situ within recombinant cells since at least one component of the antibody's natural environment will not be present. Ordinarily, however, isolated antibody will be prepared by at least one purification step.

“Native antibodies” are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (V_{H}) followed by a number of constant domains. Each light chain has a variable domain at one end (V_{L}) and a constant domain at its other end; the constant domain of the light chain is aligned with the first constant domain of the heavy chain, and the light-chain variable domain is aligned with the variable domain of the heavy chain. Particular amino acid residues are believed to form an interface between the light-chain and heavy-chain variable domains.

The “variable region” or “variable domain” of an antibody refers to the amino-terminal domains of the heavy or light chain of the antibody. The variable domain of the heavy chain may be referred to as “VH.” The variable domain of the light chain may be referred to as “VL.” These domains are generally the most variable parts of an antibody and contain the antigen-binding sites.

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called hypervariable regions (HVRs) both in the light-chain and the heavy-chain variable domains. The more highly conserved portions of variable domains are called the framework regions (FR). The variable domains of native heavy and light chains each comprise four FR regions, largely adopting a beta-sheet configuration, connected by three HVRs, which form loops connecting, and in some cases forming part of, the beta-sheet structure. The HVRs in each chain are held together in close proximity by the FR regions and, with the HVRs from the other chain, contribute to the formation of the antigen-binding site of antibodies (see Kabat et al., Sequences of Proteins of Immunological Interest, Fifth Edition, National Institute of Health, Bethesda, Md. (1991)). The constant domains are not involved directly in the binding of an antibody to an antigen, but exhibit various effector functions, such as participation of the antibody in antibody-dependent cellular toxicity.

The “light chains” of antibodies (immunoglobulins) from any vertebrate species can be assigned to one of two clearly distinct types, called kappa (κ) and lambda (λ), based on the amino acid sequences of their constant domains.

Depending on the amino acid sequences of the constant domains of their heavy chains, antibodies (immunoglobulins) can be assigned to different classes. There are five major classes of immunoglobulins: IgA, IgD, IgE, IgG, and IgM, and several of these may be further divided into subclasses (isotypes), e.g., IgG₁, IgG₂, IgG₃, IgG₄, IgA₁, and IgA₂. The heavy-chain constant domains that correspond to the different classes of immunoglobulins are called α, δ, ε, γ, and μ, respectively. The subunit structures and three-dimensional configurations of different classes of immunoglobulins are well known and described generally in, for example, Abbas et al. Cellular and Mol. Immunology, 4th ed. (W.B. Saunders, Co., 2000). An antibody may be part of a larger fusion molecule, formed by covalent or non-covalent association of the antibody with one or more other proteins or peptides.

The terms “full-length antibody,” “intact antibody,” and “whole antibody” are used herein interchangeably to refer to an antibody in its substantially intact form, not antibody fragments as defined below. The terms particularly refer to an antibody with heavy chains that contain an Fc region.

A “naked antibody” for the purposes herein is an antibody that is not conjugated to a cytotoxic moiety or radiolabel.

“Antibody fragments” comprise a portion of an intact antibody, preferably comprising the antigen-binding region thereof. Examples of antibody fragments include Fab, Fab′, F(ab′)₂, and Fv fragments; diabodies; linear antibodies; single-chain antibody molecules; and multispecific antibodies formed from antibody fragments.

Papain digestion of antibodies produces two identical antigen-binding fragments, called “Fab” fragments, each with a single antigen-binding site, and a residual “Fc” fragment, whose name reflects its ability to crystallize readily. Pepsin treatment yields a F(ab′)₂ fragment that has two antigen-combining sites and is still capable of cross-linking antigen.

“Fv” is the minimum antibody fragment which contains a complete antigen-binding site. In one embodiment, a two-chain Fv species consists of a dimer of one heavy- and one light-chain variable domain in tight, non-covalent association. In a single-chain Fv (scFv) species, one heavy- and one light-chain variable domain can be covalently linked by a flexible peptide linker such that the light and heavy chains can associate in a “dimeric” structure analogous to that in a two-chain Fv species. It is in this configuration that the three HVRs of each variable domain interact to define an antigen-binding site on the surface of the VH-VL dimer. Collectively, the six HVRs confer antigen-binding specificity to the antibody. However, even a single variable domain (or half of an Fv comprising only three HVRs specific for an antigen) has the ability to recognize and bind antigen, although at a lower affinity than the entire binding site.

The Fab fragment contains the heavy- and light-chain variable domains and also contains the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody-hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

“Single-chain Fv” or “scFv” antibody fragments comprise the VH and VL domains of an antibody, wherein these domains are present in a single polypeptide chain. Generally, the scFv polypeptide further comprises a polypeptide linker between the VH and VL domains that enables the scFv to form the desired structure for antigen binding. For a review of scFv, see, e.g., Pluckthün, in The Pharmacology of Mono-clonal Antibodies, vol. 113, Rosenburg and Moore eds. (Springer-Verlag, New York: 1994), pp 269-315.

The term “diabodies” refers to antibody fragments with two antigen-binding sites, which fragments comprise a heavy-chain variable domain (VH) connected to a light-chain variable domain (VL) in the same polypeptide chain (VH-VL). By using a linker that is too short to allow pairing between the two domains on the same chain, the domains are forced to pair with the complementary domains of another chain and create two antigen-binding sites. Diabodies may be bivalent or bispecific. Diabodies are described more fully in, for example, EP 404,097; WO 1993/01161; Hudson et al., Nat. Med. 9:129-134 (2003); and Hollinger et al., PNAS USA 90: 6444-6448 (1993). Triabodies and tetrabodies are also described in Hudson et al., Nat. Med. 9:129-134 (2003).

The term “monoclonal antibody” as used herein refers to an antibody obtained from a population of substantially homogeneous antibodies, i.e., the individual antibodies comprising the population are identical except for possible mutations, e.g., naturally occurring mutations, that may be present in minor amounts. Thus, the modifier “monoclonal” indicates the character of the antibody as not being a mixture of discrete antibodies. In certain embodiments, such a monoclonal antibody typically includes an antibody comprising a polypeptide sequence that binds a target, wherein the target-binding polypeptide sequence was obtained by a process that includes the selection of a single target binding polypeptide sequence from a plurality of polypeptide sequences. For example, the selection process can be the selection of a unique clone from a plurality of clones, such as a pool of hybridoma clones, phage clones, or recombinant DNA clones. It should be understood that a selected target binding sequence can be further altered, for example, to improve affinity for the target, to humanize the target-binding sequence, to improve its production in cell culture, to reduce its immunogenicity in vivo, to create a multispecific antibody, etc., and that an antibody comprising the altered target binding sequence is also a monoclonal antibody of this invention. In contrast to polyclonal antibody preparations, which typically include different antibodies directed against different determinants (epitopes), each monoclonal antibody of a monoclonal-antibody preparation is directed against a single determinant on an antigen. In addition to their specificity, monoclonal-antibody preparations are advantageous in that they are typically uncontaminated by other immunoglobulins.

The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including, for example, the hybridoma method (e.g., Kohler and Milstein., Nature, 256:495-97 (1975); Hongo et al., Hybridoma, 14 (3): 253-260 (1995), Harlow et al., Antibodies: A Laboratory Manual, (Cold Spring Harbor Laboratory Press, 2^{nd} ed. 1988); Hammerling et al., in: Monoclonal Antibodies and T-Cell Hybridomas 563-681 (Elsevier, N.Y., 1981)), recombinant DNA methods (see, e.g., U.S. Pat. No. 4,816,567), phage-display technologies (see, e.g., Clackson et al., Nature, 352: 624-628 (1991); Marks et al., J. Mol. Biol. 222: 581-597 (1992); Sidhu et al., J. Mol. Biol. 338(2): 299-310 (2004); Lee et al., J. Mol. Biol. 340(5): 1073-1093 (2004); Fellouse, PNAS USA 101(34): 12467-12472 (2004); and Lee et al., J. Immunol. Methods 284(1-2): 119-132 (2004), and technologies for producing human or human-like antibodies in animals that have parts or all of the human immunoglobulin loci or genes encoding human immunoglobulin sequences (see, e.g., WO 1998/24893; WO 1996/34096; WO 1996/33735; WO 1991/10741; Jakobovits et al., PNAS USA 90: 2551 (1993); Jakobovits et al., Nature 362: 255-258 (1993); Bruggemann et al., Year in Immunol. 7:33 (1993); U.S. Pat. Nos. 5,545,807; 5,545,806; 5,569,825; 5,625,126; 5,633,425; and 5,661,016; Marks et al., Bio/Technology 10: 779-783 (1992); Lonberg et al., Nature 368: 856-859 (1994); Morrison, Nature 368: 812-813 (1994); Fishwild et al., Nature Biotechnol. 14: 845-851 (1996); Neuberger, Nature Biotechnol. 14: 826 (1996); and Lonberg and Huszar, Intern. Rev. Immunol 13: 65-93 (1995).

The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (e.g., U.S. Pat. No. 4,816,567 and Morrison et al., PNAS USA 81:6851-6855 (1984)). Chimeric antibodies include PRIMATIZED® antibodies wherein the antigen-binding region of the antibody is derived from an antibody produced by, e.g., immunizing macaque monkeys with the antigen of interest.

“Humanized” forms of non-human (e.g., murine) antibodies are chimeric antibodies that contain minimal sequence derived from non-human immunoglobulin. In one embodiment, a humanized antibody is a human immunoglobulin (recipient antibody) in which residues from a HVR of the recipient are replaced by residues from a HVR of a non-human species (donor antibody) such as mouse, rat, rabbit, or nonhuman primate having the desired specificity, affinity, and/or capacity. In some instances, FR residues of the human immunoglobulin are replaced by corresponding non-human residues. Furthermore, humanized antibodies may comprise residues that are not found in the recipient antibody or in the donor antibody. These modifications may be made to further refine antibody performance. In general, a humanized antibody will comprise substantially all of at least one, and typically two, variable domains, in which all or substantially all of the hypervariable loops correspond to those of a non-human immunoglobulin, and all, or substantially all, of the FRs are those of a human immunoglobulin sequence. The humanized antibody optionally will also comprise at least a portion of an immunoglobulin constant region (Fc), typically that of a human immunoglobulin. For further details, see, e.g., Jones et al., Nature 321:522-525 (1986); Riechmann et al., Nature 332:323-329 (1988); and Presta, Curr. Op. Struct. Biol. 2:593-596 (1992). See also, for example, Vaswani and Hamilton, Ann. Allergy, Asthma & Immunol. 1:105-115 (1998); Harris, Biochem. Soc. Transactions 23:1035-1038 (1995); Hurle and Gross, Curr. Op. Biotech. 5:428-433 (1994); and U.S. Pat. Nos. 6,982,321 and 7,087,409.

A “human antibody” is one which possesses an amino-acid sequence which corresponds to that of an antibody produced by a human and/or has been made using any of the techniques for making human antibodies as disclosed herein. This definition of a human antibody specifically excludes a humanized antibody comprising non-human antigen-binding residues. Human antibodies can be produced using various techniques known in the art, including phage-display libraries. Hoogenboom and Winter, J. Mol. Biol., 227:381 (1991); Marks et al., J. Mol. Biol., 222:581 (1991). Also available for the preparation of human monoclonal antibodies are methods described in Cole et al., Monoclonal Antibodies and Cancer Therapy, Alan R. Liss, p. 77 (1985); Boerner et al., J. Immunol., 147(1):86-95 (1991). See also van Dijk and van de Winkel, Curr. Opin. Pharmacol., 5: 368-74 (2001). Human antibodies can be prepared by administering the antigen to a transgenic animal that has been modified to produce such antibodies in response to antigenic challenge, but whose endogenous loci have been disabled, e.g., immunized xenomice (see, e.g., U.S. Pat. Nos. 6,075,181 and 6,150,584 regarding XENOMOUSE™ technology). See also, for example, Li et al., PNAS USA, 103:3557-3562 (2006) regarding human antibodies generated via a human B-cell hybridoma technology.

The term “hypervariable region,” “HVR,” or “HV,” when used herein refers to the regions of an antibody-variable domain which are hypervariable in sequence and/or form structurally defined loops. Generally, antibodies comprise six HVRs; three in the VH (H1, H2, H3), and three in the VL (L1, L2, L3). In native antibodies, H3 and L3 display the most diversity of the six HVRs, and H3 in particular is believed to play a unique role in conferring fine specificity to antibodies. See, e.g., Xu et al. Immunity 13:37-45 (2000); Johnson and Wu in Methods in Molecular Biology 248:1-25 (Lo, ed., Human Press, Totowa, N.J., 2003). Indeed, naturally occurring camelid antibodies consisting of a heavy chain only are functional and stable in the absence of light chain. See, e.g., Hamers-Casterman et al., Nature 363:446-448 (1993) and Sheriff et al., Nature Struct. Biol. 3:733-736 (1996).

A number of HVR delineations are in use and are encompassed herein. The HVRs that are Kabat complementarity-determining regions (CDRs) are based on sequence variability and are the most commonly used (Kabat et al., Sequences of Proteins of Immunological Interest, 5th Ed. Public Health Service, National Institutes of Health, Bethesda, Md. (1991)). Chothia refers instead to the location of the structural loops (Chothia and Lesk J. Mol. Biol. 196:901-917 (1987)). The AbM HVRs represent a compromise between the Kabat CDRs and Chothia structural loops, and are used by Oxford Molecular's AbM antibody-modeling software. The “contact” HVRs are based on an analysis of the available complex crystal structures. The residues from each of these HVRs are noted below.

| Loop | Kabat | AbM | Chothia | Contact |
|---|---|---|---|---|
| L1 | L24-L34 | L24-L34 | L26-L32 | L30-L36 |
| L2 | L50-L56 | L50-L56 | L50-L52 | L46-L55 |
| L3 | L89-L97 | L89-L97 | L91-L96 | L89-L96 |
| H1 | H31-H35B | H26-H35B | H26-H32 | H30-H35B |

(Kabat Numbering)

| | | | | |
|---|---|---|---|---|
| H1 | H31-H35 | H26-H35 | H26-H32 | H30-H35 |

(Chothia Numbering)

| | | | | |
|---|---|---|---|---|
| H2 | H50-H65 | H50-H58 | H53-H55 | H47-H58 |
| H3 | H95-H102 | H95-H102 | H96-H101 | H93-H101 |

HVRs may comprise “extended HVRs” as follows: 24-36 or 24-34 (L1), 46-56 or 50-56 (L2), and 89-97 or 89-96 (L3) in the VL, and 26-35 (H1), 50-65 or 49-65 (H2), and 93-102, 94-102, or 95-102 (H3) in the VH. The variable-domain residues are numbered according to Kabat et al., supra, for each of these extended-HVR definitions.

“Framework” or “FR” residues are those variable-domain residues other than the HVR residues as herein defined.

The expression “variable-domain residue-numbering as in Kabat” or “amino-acid-position numbering as in Kabat,” and variations thereof, refers to the numbering system used for heavy-chain variable domains or light-chain variable domains of the compilation of antibodies in Kabat et al., supra. Using this numbering system, the actual linear amino acid sequence may contain fewer or additional amino acids corresponding to a shortening of, or insertion into, a FR or HVR of the variable domain. For example, a heavy-chain variable domain may include a single amino acid insert (residue 52a according to Kabat) after residue 52 of H2 and inserted residues (e.g. residues 82a, 82b, and 82c, etc. according to Kabat) after heavy-chain FR residue 82. The Kabat numbering of residues may be determined for a given antibody by alignment at regions of homology of the sequence of the antibody with a “standard” Kabat numbered sequence.

An “affinity-matured” antibody is one with one or more alterations in one or more HVRs thereof which result in an improvement in the affinity of the antibody for antigen, compared to a parent antibody which does not possess those alteration(s). In one embodiment, an affinity-matured antibody has nanomolar or even picomolar affinities for the target antigen. Affinity-matured antibodies are produced by procedures known in the art. For example, Marks et al., Bio/Technology 10:779-783 (1992) describes affinity maturation by VH- and VL-domain shuffling. Random mutagenesis of HVR and/or framework residues is described by, for example: Barbas et al. Proc Nat. Acad. Sci. USA 91:3809-3813 (1994); Schier et al. Gene 169:147-155 (1995); Yelton et al. J. Immunol. 155:1994-2004 (1995); Jackson et al., J. Immunol. 154(7):3310-9 (1995); and Hawkins et al, J. Mol. Biol. 226:889-896 (1992).

“Growth-inhibitory” antibodies are those that prevent or reduce proliferation of a cell expressing an antigen to which the antibody binds.

Antibodies that “induce apoptosis” are those that induce programmed cell death, as determined by standard apoptosis assays, such as binding of annexin V, fragmentation of DNA, cell shrinkage, dilation of endoplasmic reticulum, cell fragmentation, and/or formation of membrane vesicles (called apoptotic bodies).

Antibody “effector functions” refer to those biological activities attributable to the Fc region (a native-sequence Fc region or amino-acid-sequence-variant Fc region) of an antibody, and vary with the antibody isotype. Examples of antibody effector functions include: C1q binding and complement-dependent cytotoxicity (CDC); Fc-receptor binding; antibody-dependent cell-mediated cytotoxicity (ADCC); phagocytosis; down-regulation of cell-surface receptors (e.g. B-cell receptor); and B-cell activation.

The term “Fc region” herein is used to define a C-terminal region of an immunoglobulin heavy chain, including native-sequence Fc regions and variant Fc regions. Although the boundaries of the Fc region of an immunoglobulin heavy chain might vary, the human IgG heavy-chain Fc region is usually defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue.

Unless indicated otherwise herein, the numbering of the residues in an immunoglobulin heavy chain is that of the EU index as in Kabat et al., supra. The “EU index as in Kabat” refers to the residue numbering of the human IgG1 EU antibody.

A “functional Fc region” possesses an “effector function” of a native-sequence Fc region. Exemplary “effector functions” include C1q binding; CDC; Fc-receptor binding; ADCC; phagocytosis; down-regulation of cell-surface receptors (e.g. B-cell receptor; BCR), etc. Such effector functions generally require the Fc region to be combined with a binding domain (e.g. an antibody-variable domain) and can be assessed using various assays as disclosed, for example, in definitions herein.

A “native-sequence Fc region” comprises an amino acid sequence identical to the amino acid sequence of an Fc region found in nature. Native-sequence human Fc regions include a native-sequence human IgG1 Fc region (non-A and A allotypes); native-sequence human IgG2 Fc region; native-sequence human IgG3 Fc region; and native-sequence human IgG4 Fc region, as well as naturally occurring variants thereof.

A “variant Fc region” comprises an amino acid sequence which differs from that of a native-sequence Fc region by virtue of at least one amino acid modification, preferably one or more amino acid substitution(s). Preferably, the variant Fc region has at least one amino acid substitution compared to a native-sequence Fc region or to the Fc region of a parent polypeptide, e.g. from about one to about ten amino acid substitutions, and preferably from about one to about five amino acid substitutions in a native-sequence Fc region or in the Fc region of the parent polypeptide. The variant Fc region herein will preferably possess at least about 80% homology with a native-sequence Fc region and/or with an Fc region of a parent polypeptide, and most preferably at least about 90% homology therewith, more preferably at least about 95% homology therewith.

The term “Fc-region-comprising antibody” refers to an antibody that comprises an Fc region. The C-terminal lysine (residue 447 according to the EU numbering system) of the Fc region may be removed, for example, during purification of the antibody or by recombinant engineering the nucleic acid encoding the antibody. Accordingly, a composition comprising an antibody having an Fc region according to this invention can comprise an antibody with K447, with all K447 removed, or a mixture of antibodies with and without the K447 residue.

“Fc receptor” or “FcR” describes a receptor that binds to the Fc region of an antibody. In some embodiments, an FcR is a native-human FcR. In some embodiments, an FcR is one which binds an IgG antibody (a gamma receptor) and includes receptors of the FcγRI, FcγRII, and FcγRIII subclasses, including allelic variants and alternatively spliced forms of those receptors. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain. (see, e.g., Daëron, Annu. Rev. Immunol. 15:203-234 (1997)). FcRs are reviewed, for example, in Ravetch and Kinet, Annu. Rev. Immunol 9:457-92 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein.

The term “Fc receptor” or “FcR” also includes the neonatal receptor, FcRn, which is responsible for the transfer of maternal IgGs to the fetus (Guyer et al., J. Immunol. 117:587 (1976) and Kim et al., J. Immunol. 24:249 (1994)) and regulation of homeostasis of immunoglobulins. Methods of measuring binding to FcRn are known (see, e.g., Ghetie and Ward, Immunology Today, 18 (12):592-8 (1997); Ghetie et al., Nature Biotechnology, 15 (7):637-40 (1997); Hinton et al., J. Biol. Chem., 279(8):6213-6 (2004); WO 2004/92219 (Hinton et al.).

Binding to human FcRn in vivo and serum half-life of human FcRn high-affinity binding polypeptides can be assayed, e.g., in transgenic mice or transfected human cell lines expressing human FcRn, or in primates to which the polypeptides with a variant Fc region are administered. WO 2000/42072 (Presta) describes antibody variants with improved or diminished binding to FcRs. See, also, for example, Shields et al. J. Biol. Chem. 9(2): 6591-6604 (2001).

“Human effector cells” are leukocytes which express one or more FcRs and perform effector functions. In certain embodiments, the cells express at least FcγRIII and perform ADCC effector function(s). Examples of human leukocytes which mediate ADCC include peripheral blood mononuclear cells (PBMC), natural-killer (NK) cells, monocytes, cytotoxic T cells, and neutrophils. The effector cells may be isolated from a native source, e.g., from blood.

“Antibody-dependent cell-mediated cytotoxicity” or “ADCC” refers to a form of cytotoxicity in which secreted Ig bound onto Fc receptors (FcRs) present on certain cytotoxic cells (e.g., NK cells, neutrophils, and macrophages) enables these cytotoxic effector cells to bind specifically to an antigen-bearing target cell and subsequently kill the target cell with cytotoxins. The primary cells for mediating ADCC, NK cells, express FcγRIII only, whereas monocytes express FcγRI, FcγRII, and FcγRIII. FcR expression on hematopoietic cells is summarized in Table 3 on page 464 of Ravetch and Kinet, Annu. Rev. Immunol 9:457-92 (1991). To assess ADCC activity of a molecule of interest, an in vitro ADCC assay, such as that described in U.S. Pat. Nos. 5,500,362 or 5,821,337 or U.S. Pat. No. 6,737,056 (Presta), may be performed. Useful effector cells for such assays include PBMC and NK cells. Alternatively, or additionally, ADCC activity of the molecule of interest may be assessed in vivo, e.g., in an animal model such as that disclosed in Clynes et al. PNAS (USA) 95:652-656 (1998).

“Complement-dependent cytotoxicity” or “CDC” refers to the lysis of a target cell in the presence of complement. Activation of the classical complement pathway is initiated by the binding of the first component of the complement system (C1q) to antibodies (of the appropriate subclass), which are bound to their cognate antigen. To assess complement activation, a CDC assay, e.g. as described in Gazzano-Santoro et al., J. Immunol. Methods 202:163 (1996), may be performed. Polypeptide variants with altered Fc region amino acid sequences (polypeptides with a variant Fc region) and increased or decreased C1q binding capability are described, e.g., in U.S. Pat. No. 6,194,551B1 and WO 1999/51642. See, also, e.g., Idusogie et al. J. Immunol. 164: 4178-4184 (2000).

“Binding affinity” generally refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (Kd). Affinity can be measured by common methods known in the art, including those described herein. Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present invention. Specific illustrative and exemplary embodiments for measuring binding affinity are described in the following.

In one embodiment, the “Kd” or “Kd value” according to this invention is measured by a radiolabeled antigen-binding assay (RIA) performed with the Fab version of an antibody of interest and its antigen as described by the following assay. Solution-binding affinity of Fabs for antigen is measured by equilibrating Fab with a minimal concentration of ¹²⁵I)-labeled antigen in the presence of a titration series of unlabeled antigen, then capturing bound antigen with an anti-Fab antibody-coated plate (see, e.g., Chen et al., J. Mol. Biol. 293:865-881 (1999)). To establish conditions for the assay, microtiter plates (DYNEX Technologies, Inc.) are coated overnight with 5 μg/ml of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23° C.). In a non-adsorbent plate (Nunc #269620), 100 pM or 26 pM [¹²⁵I]-antigen are mixed with serial dilutions of a Fab of interest (e.g., consistent with assessment of the anti-VEGF antibody, Fab-12, in Presta et al., Cancer Res. 57:4593-4599 (1997)). The Fab of interest is then incubated overnight; however, the incubation may continue for a longer period (e.g., about 65 hours) to ensure that equilibrium is reached. Thereafter, the mixtures are transferred to the capture plate for incubation at room temperature (e.g., for one hour). The solution is then removed and the plate washed eight times with 0.1% TWEEN-20™ surfactant in PBS. When the plates have dried, 150 μl/well of scintillant (MICROSCINT-20™; Packard) is added, and the plates are counted on a TOPCOUNT™ gamma counter (Packard) for ten minutes. Concentrations of each Fab that give less than or equal to 20% of maximal binding are chosen for use in competitive binding assays.

According to another embodiment, the Kd or Kd value is measured by using surface-plasmon resonance assays using a BIACORE®-2000 or a BIACORE®-3000 instrument (BIAcore, Inc., Piscataway, N.J.) at 25° C. with immobilized antigen CM5 chips at ˜10 response units (RU). Briefly, carboxymethylated dextran biosensor chips (CM5, BIAcore Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 μg/ml (−0.2 μM) before injection at a flow rate of 5 μl/minute to achieve approximately ten response units (RU) of coupled protein. Following the injection of antigen, 1 M ethanolamine is injected to block unreacted groups. For kinetics measurements, two-fold serial dilutions of Fab (0.78 nM to 500 nM) are injected in PBS with 0.05% TWEEN 20™ surfactant (PBST) at 25° C. at a flow rate of approximately 25 μl/min. Association rates (kₒₙ) and dissociation rates (k_{off}) are calculated using a simple one-to-one Langmuir binding model (BIAcore® Evaluation Software version 3.2) by simultaneously fitting the association and dissociation sensorgrams. The equilibrium dissociation constant (Kd) is calculated as the ratio k_{off}/kₒₙ. See, e.g., Chen et al., J. Mol. Biol. 293:865-881 (1999). If the on-rate exceeds 10⁶ M⁻¹s⁻¹ by the surface-plasmon resonance assay above, then the on-rate can be determined by using a fluorescent quenching technique that measures the increase or decrease in fluorescence-emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a spectrometer, such as a stop-flow-equipped spectrophotometer (Aviv Instruments) or a 8000-series SLM-AMINCO™ spectrophotometer (ThermoSpectronic) with a stirred cuvette.

An “on-rate,” “rate of association,” “association rate,” or “kₒₙ” according to this invention can also be determined as described above using a BIACORE®-2000 or a BIACORE®-3000 system (BIAcore, Inc., Piscataway, N.J.).

The term “substantially similar” or “substantially the same,” as used herein, denotes a sufficiently high degree of similarity between two numeric values (for example, one associated with an antibody of the invention and the other associated with a reference/comparator antibody), such that one of skill in the art would consider the difference between the two values to be of little or no biological and/or statistical significance within the context of the biological characteristic measured by said values (e.g., Kd values). The difference between said two values is, for example, less than about 50%, less than about 40%, less than about 30%, less than about 20%, and/or less than about 10% as a function of the reference/comparator value.

The phrase “substantially reduced,” or “substantially different,” as used herein, denotes a sufficiently high degree of difference between two numeric values (generally one associated with a molecule and the other associated with a reference/comparator molecule) such that one of skill in the art would consider the difference between the two values to be of statistical significance within the context of the biological characteristic measured by said values (e.g., Kd values). The difference between said two values is, for example, greater than about 10%, greater than about 20%, greater than about 30%, greater than about 40%, and/or greater than about 50% as a function of the value for the reference/comparator molecule.

In certain embodiments, the humanized antibody useful herein further comprises amino acid alterations in the IgG Fc and exhibits increased binding affinity for human FcRn over an antibody having wild-type IgG Fc, by at least 60 fold, at least 70 fold, at least 80 fold, more preferably at least 100 fold, preferably at least 125 fold, even more preferably at least 150 fold to about 170 fold.

A “disorder” or “disease” is any condition that would benefit from treatment with a substance/molecule or method of the invention. This includes chronic and acute disorders or diseases including those pathological conditions which predispose the mammal to the disorder in question. Non-limiting examples of disorders to be treated herein include malignant and benign tumors; non-leukemias and lymphoid malignancies; neuronal, glial, astrocytal, hypothalamic and other glandular, macrophagal, epithelial, stromal and blastocoelic disorders; and inflammatory, immunologic and other angiogenesis-related disorders.

The terms “cell proliferative disorder” and “proliferative disorder” refer to disorders that are associated with some degree of abnormal cell proliferation. In one embodiment, the cell proliferative disorder is cancer. In one embodiment, the cell proliferative disorder is angiogenesis.

“Tumor”, as used herein, refers to all neoplastic cell growth and proliferation, whether malignant or benign, and all pre-cancerous and cancerous cells and tissues. The terms “cancer”, “cancerous”, “cell proliferative disorder”, “proliferative disorder” and “tumor” are not mutually exclusive as referred to herein.

The terms “cancer” and “cancerous” refer to or describe the physiological condition in mammals that is typically characterized by unregulated cell proliferation. Examples of cancer include but are not limited to, carcinoma, lymphoma, blastoma, sarcoma, and leukemia. More particular examples of such cancers include squamous cell cancer, lung cancer (including small-cell lung cancer, non-small cell lung cancer, adenocarcinoma of the lung, and squamous carcinoma of the lung), cancer of the peritoneum, hepatocellular cancer, gastric or stomach cancer (including gastrointestinal cancer), pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney or renal cancer, liver cancer, prostate cancer, vulval cancer, thyroid cancer, hepatic carcinoma and various types of head and neck cancer, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); Hairy cell leukemia; chronic myeloblastic leukemia; and post-transplant lymphoproliferative disorder (PTLD), as well as abnormal vascular proliferation associated with phakomatoses, edema (such as that associated with brain tumors), and Meigs' syndrome.

The term “anti-neoplastic composition” or “anti-cancer composition” or “anti-cancer agent” refers to a composition useful in treating cancer comprising at least one active therapeutic agent, e.g., “anti-cancer agent.” Examples of therapeutic agents (anti-cancer agents) include, but are limited to, e.g., chemotherapeutic agents, growth inhibitory agents, cytotoxic agents, agents used in radiation therapy, anti-angiogenesis agents, anti-lymphangiogenesis agents, apoptotic agents, anti-tubulin agents, and other-agents to treat cancer, such as anti-HER-2 antibodies, anti-CD20 antibodies, an epidermal growth factor receptor (EGFR) antagonist (e.g., a tyrosine kinase inhibitor), HER1/EGFR inhibitor (e.g., erlotinib (Tarceva™), platelet derived growth factor inhibitors (e.g., Gleevec™ (Imatinib Mesylate)), a COX-2 inhibitor (e.g., celecoxib), interferons, cytokines, antagonists (e.g., neutralizing antibodies) that bind to one or more of the following targets ErbB2, ErbB3, ErbB4, PDGFR-beta, BlyS, APRIL, BCMA VEGF, or VEGF receptor(s), TRAIL/Apo2, and other bioactive and organic chemical agents, etc. Combinations thereof are also included in the invention.

An “angiogenic factor or agent” is a growth factor or its receptor which is involved in stimulating the development of blood vessels, e.g., promote angiogenesis, endothelial cell growth, stability of blood vessels, and/or vasculogenesis, etc. For example, angiogenic factors, include, but are not limited to, e.g., VEGF and members of the VEGF family and their receptors (VEGF-B, VEGF-C, VEGF-D, VEGFR1, VEGFR2 and VEGFR3), PlGF, PDGF family, fibroblast growth factor family (FGFs), TIE ligands (Angiopoietins, ANGPT1, ANGPT2), TIE1, TIE2, ephrins, Bv8, Delta-like ligand 4 (DLL4), Del-1, fibroblast growth factors: acidic (aFGF) and basic (bFGF), FGF4, FGF9, BMP9, BMP10, Follistatin, Granulocyte colony-stimulating factor (G-CSF), GM-CSF, Hepatocyte growth factor (HGF) /scatter factor (SF), Interleukin-8 (IL-8), CXCL12, Leptin, Midkine, neuropilins, NRP1, NRP2, Placental growth factor, Platelet-derived endothelial cell growth factor (PD-ECGF), Platelet-derived growth factor, especially PDGF-BB, PDGFR-alpha, or PDGFR-beta, Pleiotrophin (PTN), Progranulin, Proliferin, Transforming growth factor-alpha (TGF-alpha), Transforming growth factor-beta (TGF-beta), Tumor necrosis factor-alpha (TNF-alpha), Alk1, CXCR4, Notch1, Notch4, Sema3A, Sema3C, Sema3F, Robo4, etc. It would further include factors that promote angiogenesis, such as ESM1 and Perlecan. It would also include factors that accelerate wound healing, such as growth hormone, insulin-like growth factor-I (IGF-I), VIGF, epidermal growth factor (EGF), EGF-like domain, multiple 7 (EGFL7), CTGF and members of its family, and TGF-alpha and TGF-beta. See, e.g., Klagsbrun and D'Amore (1991) Annu. Rev. Physiol. 53:217-39; Streit and Detmar (2003) Oncogene 22:3172-3179; Ferrara & Alitalo (1999) Nature Medicine 5(12):1359-1364; Tonini et al. (2003) Oncogene 22:6549-6556 (e.g., Table 1 listing known angiogenic factors); and, Sato (2003) Int. J. Clin. Oncol. 8:200-206.

The term “VEGF” as used herein refers to the 165-amino acid human vascular endothelial cell growth factor and related 121-, 189-, and 206-amino acid human vascular endothelial cell growth factors, as described by Leung et al. Science, 246:1306 (1989), and Houck et al. Mol. Endocrin., 5:1806 (1991), together with the naturally occurring allelic and processed forms thereof. The term “VEGF” also refers to VEGFs from non-human species such as mouse, rat or primate. Sometimes the VEGF from a specific species are indicated by terms such as hVEGF for human VEGF, mVEGF for murine VEGF, and etc. The term “VEGF” is also used to refer to truncated forms of the polypeptide comprising amino acids 8 to 109 or 1 to 109 of the 165-amino acid human vascular endothelial cell growth factor. Reference to any such forms of VEGF may be identified in the present application, e.g., by “VEGF (8-109),” “VEGF (1-109)” or “VEGF₁₆₅.” The amino acid positions for a “truncated” native VEGF are numbered as indicated in the native VEGF sequence. For example, amino acid position 17 (methionine) in truncated native VEGF is also position 17 (methionine) in native VEGF. The truncated native VEGF has binding affinity for the KDR and Flt-1 receptors comparable to native VEGF. According to a preferred embodiment, the VEGF is a human VEGF.

A “VEGF antagonist” refers to a molecule capable of neutralizing, blocking, inhibiting, abrogating, reducing or interfering with VEGF activities including its binding to VEGF or one or more VEGF receptors or the nucleic acid encoding them. Preferrably, the VEGF antagonist binds VEGF or a VEGF receptor. VEGF antagonists include anti-VEGF antibodies and antigen-binding fragments thereof, polypeptides that bind VEGF and VEGF receptors and block ligand-receptor interaction (e.g., immunoadhesins, peptibodies), anti-VEGF receptor antibodies and VEGF receptor antagonists such as small molecule inhibitors of the VEGFR tyrosine kinases, aptamers that bind VEGF and nucleic acids that hybridize under stringent conditions to nucleic acid sequences that encode VEGF or VEGF receptor (e.g., RNAi). According to one preferred embodiment, the VEGF antagonist binds to VEGF and inhibits VEGF-induced endothelial cell proliferation in vitro. According to one preferred embodiment, the VEGF antagonist binds to VEGF or a VEGF receptor with greater affinity than a non-VEGF or non-VEGF receptor. According to one preferred embodiment, the VEG antagonist binds to VEGF or a VEGF receptor with a Kd of between 1 uM and 1 pM. According to another preferred embodiment, the VEGF antagonist binds to VEGF or a VEGF receptor between 500 nM and 1 pM.

According a preferred embodiment, the VEGF antagonist is selected from a polypeptide such as an antibody, a peptibody, an immunoadhesin, a small molecule or an aptamer. In a preferred embodiment, the antibody is an anti-VEGF antibody such as the AVASTIN® antibody or an anti-VEGF receptor antibody such as an anti-VEGFR2 or an anti-VEGFR3 antibody. Other examples of VEGF antagonists include: VEGF-Trap, Mucagen, PTK787, SU11248, AG-013736, Bay 439006 (sorafenib), ZD-6474, CP632, CP-547632, AZD-2171, CDP-171, SU-14813, CHIR-258, AEE-788, SB786034, BAY579352, CDP-791, EG-3306, GW-786034, RWJ-417975/CT6758 and KRN-633.

An “anti-VEGF antibody” is an antibody that binds to VEGF with sufficient affinity and specificity. Preferably, the anti-VEGF antibody of the invention can be used as a therapeutic agent in targeting and interfering with diseases or conditions wherein the VEGF activity is involved. An anti-VEGF antibody will usually not bind to other VEGF homologues such as VEGF-B or VEGF-C, nor other growth factors such as PlGF, PDGF or bFGF. A preferred anti-VEGF antibody is a monoclonal antibody that binds to the same epitope as the monoclonal anti-VEGF antibody A4.6.1 produced by hybridoma ATCC HB 10709. More preferably the anti-VEGF antibody is a recombinant humanized anti-VEGF monoclonal antibody generated according to Presta et al. (1997) Cancer Res. 57:4593-4599, including but not limited to the antibody known as bevacizumab (BV; Avastin®). According to another embodiment, anti-VEGF antibodies that can be used include, but are not limited to the antibodies disclosed in WO 2005/012359. According to one embodiment, the anti-VEGF antibody comprises the variable heavy and variable light region of any one of the antibodies disclosed in FIGS. 24, 25, 26, 27 and 29 of WO 2005/012359 (e.g., G6, G6-23, G6-31, G6-23.1, G6-23.2, B20, B20-4 and B20.4.1). In another preferred embodiment, the anti-VEGF antibody known as ranibizumab is the VEGF antagonist administered for ocular disease such as diabetic neuropathy and AMD.

The anti-VEGF antibody “Bevacizumab (BV)”, also known as “rhuMAb VEGF” or “Avastin®”, is a recombinant humanized anti-VEGF monoclonal antibody generated according to Presta et al. (1997) Cancer Res. 57:4593-4599. It comprises mutated human IgG1 framework regions and antigen-binding complementarity-determining regions from the murine anti-hVEGF monoclonal antibody A.4.6.1 that blocks binding of human VEGF to its receptors. Approximately 93% of the amino acid sequence of Bevacizumab, including most of the framework regions, is derived from human IgG1, and about 7% of the sequence is derived from the murine antibody A4.6.1. Bevacizumab has a molecular mass of about 149,000 daltons and is glycosylated. Other anti-VEGF antibodies include the antibodies described in U.S. Pat. No. 6,884,879 and WO 2005/044853.

The anti-VEGF antibody Ranibizumab or the LUCENTIS® antibody or rhuFab V2 is a humanized, affinity-matured anti-human VEGF Fab fragment. Ranibizumab is produced by standard recombinant technology methods in Escherichia coli expression vector and bacterial fermentation. Ranibizumab is not glycosylated and has a molecular mass of ˜48,000 daltons. See WO98/45331 and US20030190317.

Dysregulation of angiogenesis can lead to abnormal angiogenesis, i.e., when excessive, insufficient, or otherwise inappropriate growth of new blood vessels (e.g., the location, timing or onset of the angiogenesis being undesired from a medical standpoint) in a diseased state or such that it causes a diseased state, i.e., an angiogenic disorder. Excessive, inappropriate or uncontrolled angiogenesis occurs when there is new blood vessel growth that contributes to the worsening of the diseased state or causes a diseased state. The new blood vessels can feed the diseased tissues, destroy normal tissues, and in the case of cancer, the new vessels can allow tumor cells to escape into the circulation and lodge in other organs (tumor metastases). Disease states involving abnormal angiogenesis (i.e., angiogenic disorders) include both non-neoplastic and neoplastic conditions including, e.g., cancer, especially vascularized solid tumors and metastatic tumors (including colon cancer, breast cancer, lung cancer (especially small-cell lung cancer), brain cancer (especially glioblastoma) or prostate cancer), undesired or aberrant hypertrophy, arthritis, rheumatoid arthritis (RA), inflammatory bowel disease or IBD (Crohn's disease and ulcerative colitis), psoriasis, psoriatic plaques, sarcoidosis, atherosclerosis, atherosclerotic plaques, diabetic and other proliferative retinopathies including retinopathy of prematurity, retrolental fibroplasia, neovascular glaucoma, age-related macular degeneration, diabetic macular edema, corneal neovascularization, corneal graft neovascularization, corneal graft rejection, retinal/choroidal neovascularization, neovascularization of the anterior surface of the iris (rubeosis), ocular neovascular disease, vascular restenosis, arteriovenous malformations (AVM), meningioma, hemangioma, angiofibroma, thyroid hyperplasias (including Grave's disease), chronic inflammation, lung inflammation, acute lung injury/ARDS, sepsis, primary pulmonary hypertension, malignant pulmonary effusions, cerebral edema (e.g., associated with acute stroke/closed head injury/trauma), synovial inflammation, myositis ossificans, hypertropic bone formation, osteoarthritis (OA), refractory ascites, polycystic ovarian disease, endometriosis, 3rd spacing of fluid diseases (pancreatitis, compartment syndrome, burns, bowel disease), uterine fibroids, premature labor, chronic inflammation such as IBD, renal allograft rejection, inflammatory bowel disease, nephrotic syndrome, undesired or aberrant tissue mass growth (non-cancer), hemophilic joints, hypertrophic scars, inhibition of hair growth, Osler-Weber syndrome, pyogenic granuloma retrolental fibroplasias, scleroderma, trachoma, vascular adhesions, synovitis, dermatitis, preeclampsia, ascites, pericardial effusion (such as that associated with pericarditis), and pleural effusion.

As used herein, “treatment” refers to clinical intervention in an attempt to alter the natural course of the individual or cell being treated, and can be performed either for prophylaxis or during the course of clinical pathology. Desirable effects of treatment include preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, antibodies of the invention are used to delay development of a disease or disorder.

An “effective amount” refers to an amount effective, at dosages and for periods of time necessary, to achieve the desired therapeutic or prophylactic result.

A “therapeutically effective amount” of a substance/molecule of the invention, agonist or antagonist may vary according to factors such as the disease state, age, sex, and weight of the individual, and the ability of the substance/molecule, agonist or antagonist to elicit a desired response in the individual. A therapeutically effective amount is also one in which any toxic or detrimental effects of the substance/molecule, agonist or antagonist are outweighed by the therapeutically beneficial effects. The term “therapeutically effective amount” refers to an amount of an antibody, polypeptide or antagonist of this invention effective to “treat” a disease or disorder in a mammal (aka patient). In the case of cancer, the therapeutically effective amount of the drug can reduce the number of cancer cells; reduce the tumor size or weight; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. To the extent the drug can prevent growth and/or kill existing cancer cells, it can be cytostatic and/or cytotoxic. In one embodiment, the therapeutically effective amount is a growth inhibitory amount. In another embodiment, the therapeutically effective amount is an amount that extends the survival of a patient. In another embodiment, the therapeutically effective amount is an amount that improves progression free survival of a patient.

A “prophylactically effective amount” refers to an amount effective, at dosages and for periods of time necessary, to achieve the desired prophylactic result. Typically but not necessarily, since a prophylactic dose is used in subjects prior to or at an earlier stage of disease, the prophylactically effective amount is less than the therapeutically effective amount.

The term “cytotoxic agent” as used herein refers to a substance that inhibits or prevents the function of cells and/or causes destruction of cells. The term is intended to include radioactive isotopes (e.g., At²¹¹, I¹³¹, I¹²⁵, Y⁹⁰, R¹⁸⁶, Re¹⁸⁸, Sm¹⁵³, Bi²¹², P³² and radioactive isotopes of Lu), chemotherapeutic agents e.g. methotrexate, adriamicin, vinca alkaloids (vincristine, vinblastine, etoposide), doxorubicin, melphalan, mitomycin C, chlorambucil, daunorubicin or other intercalating agents, enzymes and fragments thereof such as nucleolytic enzymes, antibiotics, and toxins such as small molecule toxins or enzymatically active toxins of bacterial, fungal, plant or animal origin, including fragments and/or variants thereof, and the various antitumor or anti-cancer agents disclosed below. Other cytotoxic agents are described below. A tumoricidal agent causes destruction of tumor cells.

A “chemotherapeutic agent” is a chemical compound useful in the treatment of cancer. Examples of chemotherapeutic agents include alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); delta-9-tetrahydrocannabinol (dronabinol, MARINOL®); beta-lapachone; lapachol; colchicines; betulinic acid; a camptothecin (including the synthetic analogue topotecan (HYCAMTIN®), CPT-11 (irinotecan, CAMPTOSAR®), acetylcamptothecin, scopolectin, and 9-aminocamptothecin); bryostatin; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gamma1I and calicheamicin omegaI1 (see, e.g., Agnew, Chem. Intl. Ed. Engl., 33: 183-186 (1994)); dynemicin, including dynemicin A; an esperamicin; as well as neocarzinostatin chromophore and related chromoprotein enediyne antiobiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine (ELDISINE®, FILDESIN®); dacarbazine; mannomustine; mitobronitol; mitolactol; pipobroman; gacytosine; arabinoside (“Ara-C”); thiotepa; taxoids, e.g., TAXOL® paclitaxel (Bristol-Myers Squibb Oncology, Princeton, N.J.), ABRAXANE™ Cremophor-free, albumin-engineered nanoparticle formulation of paclitaxel (American Pharmaceutical Partners, Schaumberg, Ill.), and TAXOTERE® doxetaxel (Rhone-Poulenc Rorer, Antony, France); chloranbucil; gemcitabine (GEMZAR®); 6-thioguanine; mercaptopurine; methotrexate; platinum analogs such as cisplatin and carboplatin; vinblastine (VELBAN®); platinum; etoposide (VP-16); ifosfamide; mitoxantrone; vincristine (ONCOVIN®); oxaliplatin; leucovovin; vinorelbine (NAVELBINE®); novantrone; edatrexate; daunomycin; aminopterin; ibandronate; topoisomerase inhibitor RFS 2000; difluoromethylornithine (DMFO); retinoids such as retinoic acid; capecitabine (XELODA®); pharmaceutically acceptable salts, acids or derivatives of any of the above; as well as combinations of two or more of the above such as CHOP, an abbreviation for a combined therapy of cyclophosphamide, doxorubicin, vincristine, and prednisolone, and FOLFOX, an abbreviation for a treatment regimen with oxaliplatin (ELOXATIN™) combined with 5-FU and leucovovin. Additional chemotherapeutic agents include the cytotoxic agents useful as antibody drug conjugates, such as maytansinoids (DMI, for example) and the auristatins MMAE and MMAF, for example.

“Chemotherapeutic agents” also include “anti-hormonal agents” that act to regulate, reduce, block, or inhibit the effects of hormones that can promote the growth of cancer, and are often in the form of systemic, or whole-body treatment. They may be hormones themselves. Examples include anti-estrogens and selective estrogen receptor modulators (SERMs), including, for example, tamoxifen (including NOLVADEX® tamoxifen), EVISTA® raloxifene, droloxifene, 4-hydroxytamoxifen, trioxifene, keoxifene, LY117018, onapristone, and FARESTON® toremifene; anti-progesterones; estrogen receptor down-regulators (ERDs); agents that function to suppress or shut down the ovaries, for example, leutinizing hormone-releasing hormone (LHRH) agonists such as LUPRON® and ELIGARD® leuprolide acetate, goserelin acetate, buserelin acetate and tripterelin; other anti-androgens such as flutamide, nilutamide and bicalutamide; and aromatase inhibitors that inhibit the enzyme aromatase, which regulates estrogen production in the adrenal glands, such as, for example, 4(5)-imidazoles, aminoglutethimide, MEGASE® megestrol acetate, AROMASIN® exemestane, formestanie, fadrozole, RIVISOR® vorozole, FEMARA® letrozole, and ARIMIDEX® anastrozole. In addition, such definition of chemotherapeutic agents includes bisphosphonates such as clodronate (for example, BONEFOS® or OSTAC®), DIDROCAL® etidronate, NE-58095, ZOMETA® zoledronic acid/zoledronate, FOSAMAX® alendronate, AREDIA® pamidronate, SKELID® tiludronate, or ACTONEL® risedronate; as well as troxacitabine (a 1,3-dioxolane nucleoside cytosine analog); antisense oligonucleotides, particularly those that inhibit expression of genes in signaling pathways implicated in abherant cell proliferation, such as, for example, PKC-alpha, Raf, H-Ras, and epidermal growth factor receptor (EGF-R); vaccines such as THERATOPE® vaccine and gene therapy vaccines, for example, ALLOVECTIN® vaccine, LEUVECTIN® vaccine, and VAXID® vaccine; LURTOTECAN® topoisomerase 1 inhibitor; ABARELIX® rmRH; lapatinib ditosylate (an ErbB-2 and EGFR dual tyrosine kinase small-molecule inhibitor also known as GW572016); and pharmaceutically acceptable salts, acids or derivatives of any of the above.

A “growth inhibitory agent” when used herein refers to a compound or composition which inhibits growth and/or proliferation of a cell. Examples of growth inhibitory agents include agents that block cell cycle progression (at a place other than S phase), such as agents that induce G1 arrest and M-phase arrest. Classical M-phase blockers include the vincas (vincristine and vinblastine), taxanes, and topoisomerase II inhibitors such as the anthracycline antibiotic doxorubicin ((8S-cis)-10-[(3-amino-2,3,6-trideoxy-α-L-lyxo-hexapyranosyl)oxy]-7,8,9,10-tetrahydro-6,8,11-trihydroxy-8-(hydroxyacetyl)-1-methoxy-5,12-naphthacenedione), epirubicin, daunorubicin, etoposide, and bleomycin. Those agents that arrest G1 also spill over into S-phase arrest, for example, DNA alkylating agents such as tamoxifen, prednisone, dacarbazine, mechlorethamine, cisplatin, methotrexate, 5-fluorouracil, and ara-C. Further information can be found in The Molecular Basis of Cancer, Mendelsohn and Israel, eds., Chapter 1, entitled “Cell cycle regulation, oncogenes, and antineoplastic drugs” by Murakami et al. (WB Saunders: Philadelphia, 1995), especially p. 13. The taxanes (paclitaxel and docetaxel) are anticancer drugs both derived from the yew tree. Docetaxel (TAXOTERE®, Rhone-Poulenc Rorer), derived from the European yew, is a semisynthetic analogue of paclitaxel (TAXOL®, Bristol-Myers Squibb). Paclitaxel and docetaxel promote the assembly of microtubules from tubulin dimers and stabilize microtubules by preventing depolymerization, which results in the inhibition of mitosis in cells.

As used herein, the term “patient” refers to any single animal, more preferably a mammal (including such non-human animals as, for example, dogs, cats, horses, rabbits, zoo animals, cows, pigs, sheep, and non-human primates) for which treatment is desired. Most preferably, the patient herein is a human.

A “subject” herein is any single human subject, including a patient, eligible for treatment who is experiencing or has experienced one or more signs, symptoms, or other indicators of an angiogenic disorder. Intended to be included as a subject are any subjects involved in clinical research trials not showing any clinical sign of disease, or subjects involved in epidemiological studies, or subjects once used as controls. The subject may have been previously treated with an anti-cancer agent, or not so treated. The subject may be naïve to a second medicament being used when the treatment herein is started, i.e., the subject may not have been previously treated with, for example, an anti-neoplastic agent, a chemotherapeutic agent, a growth inhibitory agent, a cytotoxic agent at “baseline” (i.e., at a set point in time before the administration of a first dose of an anti-cancer in the treatment method herein, such as the day of screening the subject before treatment is commenced). Such “naïve” subjects are generally considered to be candidates for treatment with such second medicament.

The term “effective amount” refers to an amount of a medicament that is effective for treating angiogenesis disorders or lymphangiogenesis disorders including, e.g., cancer.

The term “pharmaceutical formulation” refers to a sterile preparation that is in such form as to permit the biological activity of the medicament to be effective, and which contains no additional components that are unacceptably toxic to a subject to which the formulation would be administered.

A “sterile” formulation is aseptic or free from all living microorganisms and their spores.

A “package insert” is used to refer to instructions customarily included in commercial packages of therapeutic products or medicaments, that contain information about the indications, usage, dosage, administration, contraindications, other therapeutic products to be combined with the packaged product, and/or warnings concerning the use of such therapeutic products or medicaments, etc.

A “kit” is any manufacture (e.g a package or container) comprising at least one reagent, e.g., a medicament for treatment of an angiogenic disorder, or a probe for specifically detecting a biomarker gene or protein of the invention. The manufacture is preferably promoted, distributed, or sold as a unit for performing the methods of the present invention.

For purposes of non-response to medicament(s), a subject who experiences “a clinically unacceptably high level of toxicity” from previous or current treatment with one or more medicaments experiences one or more negative side-effects or adverse events associated therewith that are considered by an experienced clinician to be significant, such as, for example, serious infections, congestive heart failure, demyelination (leading to multiple sclerosis), significant hypersensitivity, neuropathological events, high degrees of autoimmunity, a cancer such as endometrial cancer, non-Hodgkin's lymphoma, breast cancer, prostate cancer, lung cancer, ovarian cancer, or melanoma, tuberculosis (TB), etc.

By “reducing the risk of a negative side effect” is meant reducing the risk of a side effect resulting from treatment with the antagonist herein to a lower extent than the risk observed resulting from treatment of the same patient or another patient with a previously administered medicament. Such side effects include those set forth above regarding toxicity, and are preferably infection, cancer, heart failure, or demyelination.

By “correlate” or “correlating” is meant comparing, in any way, the performance and/or results of a first analysis or protocol with the performance and/or results of a second analysis or protocol. For example, one may use the results of a first analysis or protocol in carrying out a second protocols and/or one may use the results of a first analysis or protocol to determine whether a second analysis or protocol should be performed. With respect to various embodiments herein, one may use the results of an analytical assay to determine whether a specific therapeutic regimen using an anti-cancer agent, such as anti-VEGF antibody, should be performed.

### III. Methods

The present invention provides methods for identifying patients likely to be responsive to anti-cancer agents, methods for monitoring the effectiveness of anti-cancer therapy, methods for identifying patients who have an increased likelihood of undergoing metastasis, and methods for optimizing the dose of an anti-cancer agent. The methods comprise administering an imaging agent to a patient, (a) administering an imaging agent to a patient who has received at least one dose of an anti-cancer agent; (b) detecting lymph pulsation frequency in a lymph vessel associated with a tumor draining lymph node in the patient; and (c) comparing the lymph pulsation frequency to the pulsation frequency in the lymph vessel prior to treatment with the anti-cancer agent. A decrease in lymph pulsation frequency in the lymph vessel of at least about 10% identifies a patient who has an increased likelihood of being responsive to an anti-cancer agent An increase in lymph pulsation frequency in the lymph vessel of at least about 10% identifies a patient who has an increased likelihood of undergoing metastasis. A change in lymph pulsation frequency in the lymph vessel identifies the dose as an effective dose. No change in lymph pulsation frequency in the lymph vessel identifies the dose as a maximum effective dose. In some embodiments, the methods further comprise administering an effective amount of the anti-cancer agent to the patient. In some embodiments, the methods further comprise administering an effective amount of a second, third, or fourth anti-cancer agent to the patient.

A. Imaging Methods

The disclosed methods and assays provide for convenient, efficient, and potentially cost-effective means to obtain data and information useful in assessing appropriate or effective therapies for treating patients by detecting lymphatic function (e.g., lymph pulsation frequency or bulk lymph transport). The methods may be conducted using a variety of imaging agents and devices. Suitable detection methods and devices are described in, e.g., Sharma et al., Am. J. Physiol. Heart. Circ. Physiol. 292:H3109-3118 (2007); Sharma et al., Ann. N.Y. Acad. Sci. 1131:13-36 (2008), Rasmussen et al., Curr. Opin. Biotech. 20: 74-78 (2009) and in PCT Publication Nos. WO 2008//025005 and WO 2008/02500. Using the methods described herein, lymph vessels deep beneath the tissue or skin surface may be imaged, including, e.g, lymph vessels located at a depth of at least about 1 cm, 2 cm, or 3 cm below the tissue or skin surface.

The imaging agent is administered to the patient so that the agent reaches the lymph vessels associated with a tumor draining lymph node and can be detected using methods and devices known in the art. The imaging agent may be administered to the individual through any suitable means, including, e.g., a syringe or catheter and via any suitable route including, e.g., intradermally, subcutaneously, or intramuscularly. The imaging agent may be diluted in a solution such as, e.g., saline solution, to a suitable concentration. For example, the concentration of the modified imaging agent in solution may be from about 1 μM to about 400 μM, about 10 μM to about 200 μM, or about 25 μM to about 100 μM. Any suitable amount of the imaging agent may be administered. For example, the amounts administered may be from about 1 μg to about 100 μg, about 1 μg to about 75 mg, about 1 μg to about 50 mg, about 1 μg to about 25 mg, about 1 μg to about 10 mg, about 1 μg to about 5 mg, or about 1 μg is to about 1 mg.

To excite the imaging agent in the lymphatic system, an excitation light may be illuminated on the tissue surface over the targeted region of interest by an excitation light source. Examples of suitable light sources include, e.g., laser diodes, semiconductor laser diodes, gas lasers, light emitting diodes (LEDs), or combinations thereof. In some embodiments, the excitation light source is a continuous wave light source, i.e., a light source that emits a continuous intensity of light. The light source may emit light having wavelengths from about 550 nm to about 1000 nm, about 600 nm to about 950 nm, about 700 nm to about 900 nm, or about 750 nm to about 850 nm. Alternatively, the excitation light source may be a time varying light source, i.e., a light source that emits a varying intensity of light. The intensity modulation of excitation light source may be, e.g., a sinusoidal, square wave, or ramp wave modulation. In some embodiments, the excitation light source may be pulsed at certain frequencies and repetition rates. The frequency and repetition rates may also be varied with time. The time variation of the excitation light source may be about 1 to about 3 orders of magnitude of the lifetime of the imaging agents used in conjunction with the methods described herein.

Upon illumination of the tissue surface by the excitation light, the imaging agent administered to the patient emits fluorescent light. A sensor may be used to detect or sense the emissions from the fluorescent imaging agent. The sensor is preferably capable of detecting fluorescent light emitted from the fluorescent targets and detecting excitation light reflected from the medium. In an embodiment, the sensors may comprise a charge-coupled camera (CCD). Other examples of suitable sensors include without limitation, gated or non-gated electron multiplying (EM)-CCD or intensified (ICCD) cameras. The sensor may further comprise any suitable filters or polarizers necessary to measure the appropriate wavelengths of light required for fluorescent optical tomography and imaging.

In one embodiment, fluorescent emissions from the imaging agent may be continuously detected by continuously capturing or acquiring images of the emitted light from the imaging agent to create a sequence of real-time images (i.e. a movie or video) of lymph propulsion through the lymph structures. The image may be captured for a time period, e.g., from about 100 milliseconds to about 30 minutes, about 1 minute to about 20 minutes, or about 5 minutes to about 15 minutes. Moreover, the images may be captured or recorded at any suitable integration time, e.g., from about 1 millisecond to about 5 seconds, about 10 milliseconds to about 1 second, or from about 100 milliseconds to about 800 milliseconds. Accordingly, depending on the time period and the frame rate, the images collected may be anywhere from 100 images to over 1,000 images. By tracking the imaging agent as they are pumped through the lymph structures, lymph propulsion and function may be quantitatively and accurately measured. In addition, the sequence of recorded images provides a permanent optical recording of one or more packets or masses of imaging agent being propelled or trafficked through the lymph structure (e.g., lymph vessel) upon which further analysis may be performed to assess lymph function (e.g., lymph transport such as lymph pulsation frequency or bulk lymph transport). To quantify lymph pulsation frequency, a stationary target area or region of interest may be identified on a fluorescent lymph vessel. The target area or region of interest is a point along the lymph vessel at which measurements may specifically be taken. The fluorescent intensity at the specified target area or region of interest may then be measured continuously over a given period of time. As a packet of imaging agent passes through the lymph vessel, a corresponding spike or peak in fluorescent intensity may be measured. The lymph pulsation frequency of the lymph vessel may then be quantified by dividing the number of pulse measured by the measurement time period. For example, if eight intensity peaks were measured over a time period of 5 minutes, the pulse frequency would equal about 1.6 pulses/min. Thus, the above disclosed methods are a quantitative and non-invasive way to assess lymph pulsation frequency.

In some embodiments, the methods described herein may be used in conjunction with tomographic imaging.

Any suitable imaging agent known in the art can be used in the methods of the invention. Examples of suitable imaging agents, include, e.g., tricarbocyanine dyes, bis(carbocyanine) dyes, dicarbocyanine dyes, indol-containing dyes, polymethine dyes, acridines, anthraquinones, benzimidazols, indolenines, napthalimides, oxazines, oxonols, polyenes, porphins, squaraines, styryls, thiazols, xanthins, or combinations thereof. Suitable imaging agents also include, e.g., Indocyanine Green, AlexaFluor® dyes (Invitrogen); Alexa Fluor 546, Alexa Fluor 555, Alexa Fluor 568, Alexa Fluor 594, Alexa Fluor 610, Alexa Fluor 633, Alexa Fluor 635, Alexa Fluor 647, Alexa Fluor 660, Alexa Fluor 680, Alexa Fluor 700 and Alexa Fluor 750, y dyes (GE); Cy3, Cy3.5, Cy5, Cy5.5, Cy7, IRDyes (L1-Cor); IRDye700, IRDye800, Quantum dots (Invitrogen); Qdot565, Qdot585, Qdot605, Qdot625, Qdot655, Qdot705, Qdot800, AngioSense680 and 750, AngioSpark680 and 750, VivoTag 680 and 750 (V isEn), IRdye (Sigma); IRdye740, IRdye707, IRdye743, IRdye648, IRdye814, IRdye638, IRdye762, IRdye711, IRdye784, IRdye701, IRdye712, IRdye768, IRdye683, IRdye695, IRdye668, dipicolylcyanine (DIPCY), Fluorescent proteins: mCherry, IFP1.4, DsRed, HcRed, mPlum, mRFP (reviewed in Wang et al 2008), X-Sight dyes (Carestream Health); X-Sight 640, X-Sight 670, X-Sight 549 nanosphere, X-Sight 650 nanosphere, X-Sight 691 nanosphere, X-Sight 761 nanosphere, Rhodamine, Tetramethylrhodamine isothiocyanate (TRITC), DyLight (ThermoFishcer); DyLight549, DyLight594, DyLight633, DyLight649 DyLight680, DyLight750, DyLight800, Nile red, CF dyes (Biotium); CF680, CF750, CF770, XenoLight (Caliper); XenoLightCF680, XenoLightCF750, XenoLightCF770, XenoFluor680, XenoFluor750, TransFluoSpheres (Molecular Probes): 543/620, 633/720, 633/760, FluoSpheres (Molecular Probes): Orange, Red-orange, Red, Carmine, Crimson, Scarlet, Dark red

In some embodiments, the imaging agents are conjugated to a carrier, including, e.g., polyethylene glycol, methoxypoly(ethylene glycol), dextran, or albumin.

Any suitable detection device known in the art may be used in the methods described herein. Preferably, the detection device has a resolution to visualize individual lymph vessels with a minimum field of view of 1 mm, and 3) frame rate=>0.5 Hz. Typical components of suitable devices include, e.g., light source (LED, white light, Laser, etc), filters, lense(s), detector (CCD, iCCD, EMCCD, PMT, etc) and computer with frame grabber. Suitable detection devices include, e.g., fluorescent endoscopes, epifluorescent microscopes, confocal and 2-photon microscopes, macroimaging systems and whole animal imaging systems.

Other non-fluorescence-based imaging methodologies known in the art may be used in the methods described herein. In some embodiments luminescent agents conjugated to a suitable carrier or used as free agents to visualize and measure lymph pulsation and transport by luminescent imaging devises. Suitable luminescent imaging agents include, e.g., luciferase and BRET-Qdots (Kosaka et al., Contrast Media and Mol. Im. DOI: 10.1002/cmmi.395 (2010).

In some embodiments photoacoustic imaging agents are conjugated to a suitable carrier or used as free agents to visualize and measure lymph pulsation and transport by photoacoustic imaging (Song et al., Med. Phys. 36:3724-9 (2009), Erpelding et al., Radiology 256:102-10 (2010), Kim et al., Radiology 255:442-50 (2010)). Suitable photoacoustic imaging agents include, e.g., Evans blue (Song et al., Med. Phys. 36:3724-9 (2009)), methylene blue (Erpelding et al., Radiology 256:102-10 (2010)) and Indocyanine Green (Kim et al., Radiology 255:442-50 (2010)).

In some embodiments lymph vessels can be observed directly using other imaging methodology known in the art, including, for example, optical-coherence tomography (McLaughlin et al., Cancer Res. 70:2579-84 (2010)) and optical frequency domain imaging (OFDI) (Vakoc et al., Nat. Med. 15:1219-24 (2009)). Contrast agents can be used with these methods to increase detection and sensitivity.

Other non-optical based detection devices known in the art may be used in the methods described herein (Clement and Luciani Eur. Radiol. 14:1498-1507 (2004), Barrett et al. Contrast Media and Mol. Img. 1:230-245 (2006)). In some embodiments, Magnetic Resonance Imaging (MRI) agents are used visualize and measure lymph pulsation and transport by MRI (Motoyama et al., Surgery 141:736-47 (2007), Ruddel et al. Neoplasia 10:706-13 (2008) and Notohamiprodjo et al., Eur. Radiol. 19:2771-8 (2009)). Suitable imaging agents include, e.g., iron oxide particles (Motoyama et al., Surgery 141:736-47 (2007)), nanotubes (Ananta et al., Nano Lett. 9:1023-27 (2009)), gadolinium-dimeglumine (Notohamiprodjo et al., Eur. Radiol. 19:2771-8 (2009)) and gadolinium-based nanotubes (Sitharaman and Wilson J. Nanomed. 1:291-5 (2006)). The MRI agents may be conjugated to a suitable carrier or used as free agents.

In some embodiments, ultrasound-imaging agents are used to visualize and measure lymph pulsation and transport by ultrasound imaging (Curry et al., Ann. Otol Rhinol Laryngol. 118:645-50 (2009)). Suitable ultrasound-imaging agents include, e.g., perflubutane microbubbles (Sonazoid, Amersham). The ultrasound-imaging agents may be conjugated to a suitable carrier or used as free agents.

In some embodiments, radionucleoides are used to visualize and measure lymph pulsation and transport by Positron-Emission Tomography (PET), Single-Photon Emission Computed Tomography (SPECT) or time resolved radiography imaging (Weiss et al., Eur. J. Nuc. Med. Mol. 1 mg. 30:202-6 (2003), March et al., J. Nuc. Med. Tech 35:10-16 (2007)). Suitable radionucleoides include, e.g., Tc-99, F-18, Cu-64I-124, Br-76, Br77, C-11, In-111, I-123, Y-86, Cu-60, Cu-61, and Zr-89. The radionuclides may be conjugated to a suitable carrier or used as free agents.

In some embodiments, computed-tomography (CT) imaging agents are used to visualize and measure lymph pulsation and transport by computed tomography imaging (Suga et al., Radiology 230:543-552 (2004), Suga et al., Radiology 237:952-60 (2005)). Suitable CT imaging agents include, e.g., iopamidol (Suga et al., Radiology 230:543-552 (2004)). The CT imaging agents may be conjugated to a suitable carrier or used as free agents.

B. Additional Methods

In addition to the methods described herein above using imaging agents, a clinician may use any of several methods known in the art to measure the effectiveness of a particular dosage scheme of an anti-cancer agent. For example, in vivo imaging (e.g., MRI) can be used to determine the tumor size and to identify any metastases to determine relative effective responsiveness to the therapy. Dosage regimens may be adjusted to provide the optimum desired response (e.g., a therapeutic response). For example, a dose may be administered, several divided doses may be administered over time or the dose may be proportionally reduced or increased as indicated by exigencies of the therapeutic situation.

A physician having ordinary skill in the art can readily determine and prescribe the effective amount of the pharmaceutical composition required, depending on such factors as the particular type of anti-cancer agent. For example, the physician could start with doses of such anti-cancer agent, such as an anti-NRP2 antibody, an anti-VEGF-C antibody, or an anti-VEGF-A antibody, employed in the pharmaceutical composition at levels lower than that required in order to achieve the desired therapeutic effect and gradually increase the dosage until the desired effect is achieved. The effectiveness of a given dose or treatment regimen of the antagonist can be determined, for example, by assessing signs and symptoms in the patient using standard measures of efficacy.

In yet another embodiment, the subject is treated with the same anti-cancer agent, such as an anti-NRP2 antibody, an anti-VEGF-C antibody, or an anti-VEGF-A antibody at least twice. Thus, the initial and second antagonist exposures are preferably with the same antagonist, and more preferably all antagonist exposures are with the same antagonist, i.e., treatment for the first two exposures, and preferably all exposures, is with one type of anti-cancer agent, for example, an antagonist that binds to VEGF, such as an anti-VEGF antibody, e.g., all with bevacizumab.

In all the inventive methods set forth herein, the anti-cancer agent (such as an antibody that binds to VEGF) may be unconjugated, such as a naked antibody, or may be conjugated with another molecule for further effectiveness, such as, for example, to improve half-life.

One preferred anti-cancer agent herein is a chimeric, humanized, or human antibody, e.g., an anti-VEGF antibody, and preferably bevacizumab.

In another embodiment, the VEGF antagonist (e.g., an anti-VEGF antibody) is the only medicament administered to the subject.

In one embodiment, the antagonist is an anti-VEGF antibody that is administered at a dose of about 100 or 400 mg every 1, 2, 3, or 4 weeks or is administered a dose of about 1, 3, 5, 10, 15, or 20 mg/kg every 1, 2, 3, or 4 weeks. The dose may be administered as a single dose or as multiple doses (e.g., 2 or 3 doses), such as infusions.

In yet another aspect, the invention provides, after the diagnosis step, a method of determining whether to continue administering an anti-cancer agent (e.g., an anti-VEGF antibody) to a subject diagnosed with cancer comprising measuring reduction in tumor size, using imaging techniques, such as radiography and/or MRI, after administration of the antagonist a first time, measuring reduction in tumor size in the subject, using imaging techniques such as radiography and/or MRI after administration of the antagonist a second time, comparing imaging findings in the subject at the first time and at the second time, and if the score is less at the second time than at the first time, continuing administration of the antagonist.

In a still further embodiment, a step is included in the treatment method to test the subject's response to treatment after the administration step to determine that the level of response is effective to treat the angiogenic disorder. For example, a step is included to test the imaging (radiographic and/or MRI) score after administration and compare it to baseline imaging results obtained before administration to determine if treatment is effective by measuring if, and by how much, it has been changed. This test may be repeated at various scheduled or unscheduled time intervals after the administration to determine maintenance of any partial or complete remission.

In one embodiment of the invention, no other medicament than VEGF antagonist such as anti-VEGF antibody is administered to the subject to treat cancer.

In any of the methods herein, the anti-cancer agent may be administered in combination with an effective amount of a second medicament. Suitable second medicament include, for example, an anti-lymphangiogenic agent, an anti-angiogenic agent, an anti-neoplastic agent, a chemotherapeutic agent, a growth inhibitory agent, a cytotoxic agent, or combinations thereof.

All these second medicaments may be used in combination with each other or by themselves with the first medicament, so that the expression “second medicament” as used herein does not mean it is the only medicament in addition to the first medicament. Thus, the second medicament need not be a single medicament, but may constitute or comprise more than one such drug.

These second medicaments as set forth herein are generally used in the same dosages and with administration routes as used hereinbefore or about from 1 to 99% of the heretofore-employed dosages. If such second medicaments are used at all, preferably, they are used in lower amounts than if the first medicament were not present, especially in subsequent dosings beyond the initial dosing with the first medicament, so as to eliminate or reduce side effects caused thereby.

For the re-treatment methods described herein, where a second medicament is administered in an effective amount with an antagonist exposure, it may be administered with any exposure, for example, only with one exposure, or with more than one exposure. In one embodiment, the second medicament is administered with the initial exposure. In another embodiment, the second medicament is administered with the initial and second exposures. In a still further embodiment, the second medicament is administered with all exposures. It is preferred that after the initial exposure, such as of steroid, the amount of such second medicament is reduced or eliminated so as to reduce the exposure of the subject to an agent with side effects such as prednisone, prednisolone, methylprednisolone, and cyclophosphamide.

The combined administration of a second medicament includes co-administration (concurrent administration), using separate formulations or a single pharmaceutical formulation, and consecutive administration in either order, wherein preferably there is a time period while both (or all) active agents (medicaments) simultaneously exert their biological activities.

The anti-cancer agent is administered by any suitable means, including parenteral, topical, subcutaneous, intraperitoneal, intrapulmonary, intranasal, and/or intralesional administration. Parenteral infusions include intramuscular, intravenous (i.v.), intraarterial, intraperitoneal, or subcutaneous administration. Intrathecal administration is also contemplated. In addition, the anti-cancer agent may suitably be administered by pulse infusion, e.g., with declining doses of the anti-cancer agent. Preferably, the dosing is given intravenously or subcutaneously, and more preferably by intravenous infusion(s).

If multiple exposures of anti-cancer agents are provided, each exposure may be provided using the same or a different administration means. In one embodiment, each exposure is by intravenous administration. In another embodiment, each exposure is given by subcutaneous administration. In yet another embodiment, the exposures are given by both intravenous and subcutaneous administration.

In one embodiment, the anti-cancer agent such as an anti-VEGF antibody is administered as a slow intravenous infusion rather than an intravenous push or bolus. For example, a steroid such as prednisolone or methylprednisolone (e.g., about 80-120 mg i.v., more specifically about 100 mg i.v.) is administered about 30 minutes prior to any infusion of the anti-VEGF antibody. The anti-VEGF antibody is, for example, infused through a dedicated line.

For the initial dose of a multi-dose exposure to anti-VEGF antibody, or for the single dose if the exposure involves only one dose, such infusion is preferably commenced at a rate of about 50 mg/hour. This may be escalated, e.g., at a rate of about 50 mg/hour increments every about 30 minutes to a maximum of about 400 mg/hour. However, if the subject is experiencing an infusion-related reaction, the infusion rate is preferably reduced, e.g., to half the current rate, e.g., from 100 mg/hour to 50 mg/hour. Preferably, the infusion of such dose of anti-VEGF antibody (e.g., an about 1000-mg total dose) is completed at about 255 minutes (4 hours 15 min.). Optionally, the subjects receive a prophylactic treatment of acetaminophen/paracetamol (e.g., about 1 g) and diphenhydramine HCl (e.g., about 50 mg or equivalent dose of similar agent) by mouth about 30 to 60 minutes prior to the start of an infusion.

If more than one infusion (dose) of anti-VEGF antibody is given to achieve the total exposure, the second or subsequent anti-VEGF antibody infusions in this infusion embodiment are preferably commenced at a higher rate than the initial infusion, e.g., at about 100 mg/hour. This rate may be escalated, e.g., at a rate of about 100 mg/hour increments every about 30 minutes to a maximum of about 400 mg/hour. Subjects who experience an infusion-related reaction preferably have the infusion rate reduced to half that rate, e.g., from 100 mg/hour to 50 mg/hour. Preferably, the infusion of such second or subsequent dose of anti-VEGF antibody (e.g., an about 1000-mg total dose) is completed by about 195 minutes (3 hours 15 minutes).

In a preferred embodiment, the anti-cancer agent is an anti-VEGF antibody and is administered in a dose of about 0.4 to 4 grams, and more preferably the antibody is administered in a dose of about 0.4 to 1.3 grams at a frequency of one to four doses within a period of about one month. Still more preferably, the dose is about 500 mg to 1.2 grams, and in other embodiments is about 750 mg to 1.1 grams. In such aspects, the antagonist is preferably administered in two to three doses, and/or is administered within a period of about 2 to 3 weeks.

In one embodiment, the subject has never been previously administered any drug(s) to treat the cancer. In another embodiment, the subject or patient has been previously administered one or more medicaments(s) to treat the cancer. In a further embodiment, the subject or patient was not responsive to one or more of the medicaments that had been previously administered. Such drugs to which the subject may be non-responsive include, for example, anti-neoplastic agents, chemotherapeutic agents, cytotosic agents, and/or growth inhibitory agents. More particularly, the drugs to which the subject may be non-responsive include VEGF antagonists such as anti-VEGF antibodies. In a further aspect, such anti-cancer agent include an antibody or immunoadhesin, such that re-treatment is contemplated with one or more antibodies or immunoadhesins of this invention to which the subject was formerly non-responsive.

### IV. Treatment with the Anti-Cancer Agent

Once the patient population most responsive or sensitive to treatment with the antagonist has been identified, treatment with the anti-cancer agent, alone or in combination with other medicaments, results in an improvement in the cancer. For instance, such treatment may result in a reduction in tumor size or progression free survival. Moreover, treatment with the combination of an anti-cancer agent and at least one second medicament(s) preferably results in an additive, more preferably synergistic (or greater than additive) therapeutic benefit to the patient. Preferably, in this combination method the timing between at least one administration of the second medicament and at least one administration of the anti-cancer agent is about one month or less, more preferably, about two weeks or less.

It will be appreciated by one of skill in the medical arts that the exact manner of administering to said patient a therapeutically effective amount of an anti-cancer agent following a diagnosis of a patient's likely responsiveness to the anti-cancer agent will be at the discretion of the attending physician. The mode of administration, including dosage, combination with other agents, timing and frequency of administration, and the like, may be affected by the diagnosis of a patient's likely responsiveness to such anti-cancer agent, as well as the patient's condition and history. Thus, even patients diagnosed with a disorder who are predicted to be relatively insensitive to the anti-cancer agent may still benefit from treatment therewith, particularly in combination with other agents, including agents that may alter a patient's responsiveness to the anti-cancer agent.

The composition comprising an anti-cancer agent will be formulated, dosed, and administered in a fashion consistent with good medical practice. Factors for consideration in this context include the particular type of disorder being treated, the particular mammal being treated, the clinical condition of the individual patient, the cause of the angiogenic disorder, the site of delivery of the agent, possible side-effects, the type of antagonist, the method of administration, the scheduling of administration, and other factors known to medical practitioners. The effective amount of the anti-cancer agent to be administered will be governed by such considerations.

As a general proposition, the effective amount of the anti-cancer agent administered parenterally per dose will be in the range of about 20 mg to about 5000 mg, by one or more dosages. Exemplary dosage regimens for antibodies such as anti-VEGF antibodies include 100 or 400 mg every 1, 2, 3, or 4 weeks or is administered a dose of about 1, 3, 5, 10, 15, or 20 mg/kg every 1, 2, 3, or 4 weeks. The dose may be administered as a single dose or as multiple doses (e.g., 2 or 3 doses), such as infusions.

As noted above, however, these suggested amounts of anti-cancer agent are subject to a great deal of therapeutic discretion. The key factor in selecting an appropriate dose and scheduling is the result obtained, as indicated above. In some embodiments, the anti-cancer agent is administered as close to the first sign, diagnosis, appearance, or occurrence of the disorder as possible.

The anti-cancer agent is administered by any suitable means, including parenteral, topical, subcutaneous, intraperitoneal, intrapulmonary, intranasal, and/or intralesional administration. Parenteral infusions include intramuscular, intravenous, intraarterial, intraperitoneal, or subcutaneous administration. Intrathecal administration is also contemplated. In addition, the antagonist may suitably be administered by pulse infusion, e.g., with declining doses of the antagonist. Most preferably, the dosing is given by intravenous injections.

One may administer a second medicament, as noted above, with the anti-cancer agents herein. The combined administration includes co-administration, using separate formulations or a single pharmaceutical formulation, and consecutive administration in either order, wherein preferably there is a time period while both (or all) active agents simultaneously exert their biological activities.

Aside from administration of anti-cancer agents to the patient by traditional routes as noted above, the present invention includes administration by gene therapy. Such administration of nucleic acids encoding the anti-cancer agent is encompassed by the expression “administering an effective amount of an anti-cancer agent”. See, for example, WO 1996/07321 concerning the use of gene therapy to generate intracellular antibodies.

There are two major approaches to getting the nucleic acid (optionally contained in a vector) into the patient's cells; in vivo and ex vivo. For in vivo delivery the nucleic acid is injected directly into the patient, usually at the site where the antagonist is required. For ex vivo treatment, the patient's cells are removed, the nucleic acid is introduced into these isolated cells and the modified cells are administered to the patient either directly or, for example, encapsulated within porous membranes which are implanted into the patient (see, e.g. U.S. Pat. Nos. 4,892,538 and 5,283,187). There are a variety of techniques available for introducing nucleic acids into viable cells. The techniques vary depending upon whether the nucleic acid is transferred into cultured cells in vitro or in vivo in the cells of the intended host. Techniques suitable for the transfer of nucleic acid into mammalian cells in vitro include the use of liposomes, electroporation, microinjection, cell fusion, DEAE-dextran, the calcium phosphate precipitation method, etc. A commonly used vector for ex vivo delivery of the gene is a retrovirus.

The currently preferred in vivo nucleic acid transfer techniques include transfection with viral vectors (such as adenovirus, Herpes simplex I virus, or adeno-associated virus) and lipid-based systems (useful lipids for lipid-mediated transfer of the gene are DOTMA, DOPE and DC-Chol, for example). In some situations it is desirable to provide the nucleic acid source with an agent specific for the target cells, such as an antibody specific for a cell-surface membrane protein on the target cell, a ligand for a receptor on the target cell, etc. Where liposomes are employed, proteins that bind to a cell-surface membrane protein associated with endocytosis may be used for targeting and/or to facilitate uptake, e.g. capsid proteins or fragments thereof tropic for a particular cell type, antibodies for proteins that undergo internalization in cycling, and proteins that target intracellular localization and enhance intracellular half-life. The technique of receptor-mediated endocytosis is described, for example, by Wu et al., J. Biol. Chem. 262:4429-4432 (1987); and Wagner et al., PNAS USA 87:3410-3414 (1990). Gene-marking and gene-therapy protocols are described, for example, in Anderson et al., Science 256:808-813 (1992) and WO 1993/25673.

An anti-cancer agent may be combined in a pharmaceutical combination formulation, or dosing regimen as combination therapy, with at least one additional compound having anti-cancer properties. The at least one additional compound of the pharmaceutical combination formulation or dosing regimen preferably has complementary activities to the VEGF antagonist composition such that they do not adversely affect each other.

The at least one additional compound may be a chemotherapeutic agent, a cytotoxic agent, a cytokine, a growth inhibitory agent, an anti-hormonal agent, an anti-angiogenic agent, an anti-lymphangiogenic agent, and combinations thereof. Such molecules are suitably present in combination in amounts that are effective for the purpose intended. A pharmaceutical composition containing an VEGF antagonist (e.g., an anti-VEGF antibody) may also comprise a therapeutically effective amount of an anti-neoplastic agent, a chemotherapeutic agent a growth inhibitory agent, a cytotoxic agent, or combinations thereof.

In one aspect, the first compound is an anti-VEGF antibody and the at least one additional compound is a therapeutic antibody other than an anti-VEGF antibody. In one embodiment, the at least one additional compound is an antibody that binds a cancer cell surface marker. In one embodiment the at least one additional compound is an anti-HER2 antibody, trastuzumab (e.g., Herceptin®, Genentech, Inc., South San Francisco, Calif.). In one embodiment the at least one additional compound is an anti-HER2 antibody, pertuzumab (Omnitarg™, Genentech, Inc., South San Francisco, Calif., see U.S. Pat. No. 6,949,245). In an embodiment, the at least one additional compound is an antibody (either a naked antibody or an ADC), and the additional antibody is a second, third, fourth, fifth, sixth antibody or more, such that a combination of such second, third, fourth, fifth, sixth, or more antibodies (either naked or as an ADC) is efficacious in treating an angiogenic disorder.

Other therapeutic regimens in accordance with this invention may include administration of a VEGF-antagonist anti-cancer agent and, including without limitation radiation therapy and/or bone marrow and peripheral blood transplants, and/or a cytotoxic agent, a chemotherapeutic agent, or a growth inhibitory agent. In one of such embodiments, a chemotherapeutic agent is an agent or a combination of agents such as, for example, cyclophosphamide, hydroxydaunorubicin, adriamycin, doxorubincin, vincristine (ONCOVIN™), prednisolone, CHOP, CVP, or COP, or immunotherapeutics such as anti-PSCA, anti-HER2 (e.g., HERCEPTIN®, OMNITARG™). The combination therapy may be administered as a simultaneous or sequential regimen. When administered sequentially, the combination may be administered in two or more administrations. The combined administration includes coadministration, using separate formulations or a single pharmaceutical formulation, and consecutive administration in either order, wherein preferably there is a time period while both (or all) active agents simultaneously exert their biological activities.

In one embodiment, treatment with an anti-VEGF antibody involves the combined administration of an anti-cancer agent identified herein, and one or more chemotherapeutic agents or growth inhibitory agents, including coadministration of cocktails of different chemotherapeutic agents. Chemotherapeutic agents include taxanes (such as paclitaxel and docetaxel) and/or anthracycline antibiotics. Preparation and dosing schedules for such chemotherapeutic agents may be used according to manufacturer's instructions or as determined empirically by the skilled practitioner. Preparation and dosing schedules for such chemotherapy are also described in “Chemotherapy Service”, (1992) Ed., M. C. Perry, Williams & Wilkins, Baltimore, Md.

Suitable dosages for any of the above coadministered agents are those presently used and may be lowered due to the combined action (synergy) of the newly identified agent and other chemotherapeutic agents or treatments.

The combination therapy may provide “synergy” and prove “synergistic”, i.e. the effect achieved when the active ingredients used together is greater than the sum of the effects that results from using the compounds separately. A synergistic effect may be attained when the active ingredients are: (1) co-formulated and administered or delivered simultaneously in a combined, unit dosage formulation; (2) delivered by alternation or in parallel as separate formulations; or (3) by some other regimen. When delivered in alternation therapy, a synergistic effect may be attained when the compounds are administered or delivered sequentially, e.g. by different injections in separate syringes. In general, during alternation therapy, an effective dosage of each active ingredient is administered sequentially, i.e. serially, whereas in combination therapy, effective dosages of two or more active ingredients are administered together.

For the prevention or treatment of disease, the appropriate dosage of the additional therapeutic agent will depend on the type of disease to be treated, the type of antibody, the severity and course of the disease, whether the VEGF antagonist and additional agent are administered for preventive or therapeutic purposes, previous therapy, the patient's clinical history and response to the VEGF antagonist and additional agent, and the discretion of the attending physician. The VEGF antagonist and additional agent are suitably administered to the patient at one time or over a series of treatments. The VEGF antagonist is typically administered as set forth above. Depending on the type and severity of the disease, about 20 mg/m² to 600 mg/m² of the additional agent is an initial candidate dosage for administration to the patient, whether, for example, by one or more separate administrations, or by continuous infusion. One typical daily dosage might range from about or about 20 mg/m², 85 mg/m², 90 mg/m², 125 mg/m², 200 mg/m², 400 mg/m², 500 mg/m² or more, depending on the factors mentioned above. For repeated administrations over several days or longer, depending on the condition, the treatment is sustained until a desired suppression of disease symptoms occurs. Thus, one or more doses of about 20 mg/m², 85 mg/m², 90 mg/m², 125 mg/m², 200 mg/m², 400 mg/m², 500 mg/m², 600 mg/m² (or any combination thereof) may be administered to the patient. Such doses may be administered intermittently, e.g. every week or every two, three weeks, four, five, or six (e.g. such that the patient receives from about two to about twenty, e.g. about six doses of the additional agent). An initial higher loading dose, followed by one or more lower doses may be administered. However, other dosage regimens may be useful. The progress of this therapy is easily monitored by conventional techniques and assays.

### V. Pharmaceutical Formulations

Therapeutic formulations of the antagonists used in accordance with the present invention are prepared for storage by mixing the antagonist having the desired degree of purity with optional pharmaceutically acceptable carriers, excipients, or stabilizers in the form of lyophilized formulations or aqueous solutions. For general information concerning formulations, see, e.g., Gilman et al., (eds.) (1990), The Pharmacological Bases of Therapeutics, 8th Ed., Pergamon Press; A. Gennaro (ed.), Remington's Pharmaceutical Sciences, 18th Edition, (1990), Mack Publishing Co., Eastori, Pa.; Avis et al., (eds.) (1993) Pharmaceutical Dosage Forms: Parenteral Medications Dekker, New York; Lieberman et al., (eds.) (1990) Pharmaceutical Dosage Forms: Tablets Dekker, New York; and Lieberman et al., (eds.) (1990), Pharmaceutical Dosage Forms: Disperse Systems Dekker, New York, Kenneth A. Walters (ed.) (2002) Dermatological and Transdermal Formulations (Drugs and the Pharmaceutical Sciences), Vol 119, Marcel Dekker.

Acceptable carriers, excipients, or stabilizers are non-toxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™, or polyethylene glycol (PEG).

Exemplary anti-VEGF antibody formulations are described in U.S. Pat. Nos. 6,884,879. In certain embodiments anti-VEGF antibodies are formulated at 25 mg/mL in single use vials. In certain embodiments, 100 mg of the anti-VEGF antibodies are formulated in 240 mg α,α-trehalose dihydrate, 23.2 mg sodium phosphate (monobasic, monohydrate), 4.8 mg sodium phosphate (dibasic anhydrous), 1.6 mg polysorbate 20, and water for injection, USP. In certain embodiments, 400 mg of the anti-VEGF antibodies are formulated in 960 mg α,α-trehalose dihydrate, 92.8 mg sodium phosphate (monobasic, monohydrate), 19.2 mg sodium phosphate (dibasic anhydrous), 6.4 mg polysorbate 20, and water for injection, USP.

Lyophilized formulations adapted for subcutaneous administration are described, for example, in U.S. Pat. No. 6,267,958 (Andya et al.). Such lyophilized formulations may be reconstituted with a suitable diluent to a high protein concentration and the reconstituted formulation may be administered subcutaneously to the mammal to be treated herein.

Crystallized forms of the antagonist are also contemplated. See, for example, US 2002/0136719A1 (Shenoy et al.).

The formulation herein may also contain more than one active compound (a second medicament as noted above), preferably those with complementary activities that do not adversely affect each other. The type and effective amounts of such medicaments depend, for example, on the amount and type of VEGF antagonist present in the formulation, and clinical parameters of the subjects. The preferred such second medicaments are noted above.

The active ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semi-permeable matrices of solid hydrophobic polymers containing the antagonist, which matrices are in the form of shaped articles, e.g. films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid.

The formulations to be used for in vivo administration must be sterile. This is readily accomplished by filtration through sterile filtration membranes.

### VI. Kits

For use in detection of the lymph function, kits or articles of manufacture are also provided by the invention. Such kits can be used to determine if a subject with cancer will be effectively responsive to an anti-cancer agent. These kits may comprise a carrier means being compartmentalized to receive in close confinement one or more container means such as vials, tubes, and the like, each of the container means comprising one of the separate elements to be used in the method. For example, one of the container means may comprise an imaging agent.

Such kit will typically comprise the container described above and one or more other containers comprising materials desirable from a commercial and user standpoint, including buffers, diluents, filters, needles, syringes, and package inserts with instructions for use. A label may be present on the container to indicate that the composition is used for a specific application, and may also indicate directions for either in vivo or in vitro use, such as those described above.

The kits of the invention have a number of embodiments. A typical embodiment is a kit comprising a container, a label on said container, and a composition contained within said container, wherein the composition includes an imaging agent and the label on said container indicates that the composition can be used to lymph pulsation frequency, and wherein the kit includes instructions for using the imaging agent for detecting lymph pulsation frequency. The kit can further comprise a set of instructions and materials for preparing and administering the imaging agent.

Other optional components of the kit include one or more buffers (e.g., dilution buffer, etc.), other reagents such as carrier (e.g., dextran, albumin) Kits can also include instructions for interpreting the results obtained using the kit.

### EXAMPLES

The following examples are provided to illustrate, but not to limit the presently claimed invention.

### Example 1

### Materials and Methods

Animals:

Female Balb-c nude mice from Charles River Laboratory were used at 6-8 weeks of age. To establish baseline pulsation frequency and lymph node loading rates we imaged non-tumor bearing mice. In some cases mice were longitudinally imaged for pulsation, i.e., the same mouse was imaged daily over a 7-day period. Mice were anesthetized with 2% Isoflurane and maintained at 37° C. throughout experiments. A PulseOx probe was attached to the thigh (opposing injection site) to monitor heart rate before running pulsation assays.

Imaging Agent:

For all experiments fluorescent dye injections were performed with 5 mg AlexaFluor680 conjugated to dextran 70 kd (MolecularProbes) in 1 ml sterile PBS.

Lymph Pulsation Frequency Assay:

Dye was delivered using a 3/10 cc syringe with a 28½ gauge needle (Beckton Dickinson). A 15 μl bolus of dye was injected intradermally near the base of tail, 5 mm lateral to the rectum. Injection site bulges slightly then dye visibly drains through lymphatic vessels leading to inguinal lymph node within 5 minutes.

Epi-fluorescence Microscopy Pulsatile flow activity was observed using an epifluorescence microscope (Prairie Technologies) equipped with a Cy5.5 Red filter set (Chroma), Halogen light source (ExFo Excite Series 120), 4× objective lens (Olympus) and a charge coupled device (CCD) camera (S97827, Olympus). Mice were transferred to heated platform under the objective lens immediately following dye injection. Animal were positioned to lie laterally, exposing dye-filled lymph vessel from inguinal to axial lymph node. A ˜5 mm section along the vessel was identified as region of interest for imaging pulsatile lymph flow (i.e., lymph pulsation frequency). This section was then lightly covered by a poseable glass slide to provide a flat surface and reduce breathing artifact. Pulsation and heart rates were monitored (PulseOx) for stability for 10 minutes before video capture. The stage was plumbed for anesthesia and thermo-regulated to maintain animal temperature at 37° C.

Digital videos (5 minutes) of pulsation were acquired with PictureFrame software (Optronics) using time lapse settings (282 ms exposure, no inter-frame delay, 5 minutes total acquisition time). Data was analyzed off-line from recorded time lapse videos.

Bulk Lymph Transport Assay:

Dye was delivered by an infusion pump attached to a catheter implanted at the intradermal injection site near the base of the tail, 5 mm lateral to the rectum. Catheter was prepped by cutting off needle tip from 3/10 cc, 28 281/2 gauge (BD) and placing into Micro-Renathane (Braintree Scientific) tubing loaded with 75 μl of dye.

Whole animal near infared-flourescence (NIRF) imaging bulk lymph transport to inguinal node was observed using a Kodak 4000FX Pro NIRF Imaging system. After implanting a catheter, mice were transferred to the Kodak imaging platform, and the catheter was routed outside to the infusion pump.

Mice were positioned on the imaging surface on their lateral side, to capture inguinal node and vessel leading to axial node in the field of view (FOV).

Transport from injection site to inguinal node then axial node was captured using Kodak imaging software. Time lapse settings were set to: 2 second exposure, 15 second interval, 21.5 mm FOV, ex 650 nm/em700 nm, 30 min total acquisition time). Transport recording started at the same time infusion pump (5 μL/min) began. Data was analyzed off-line from recorded time-lapse images.

Image Analysis/Quantitation:

Images collected on the Kodak system were converted to tiff format using custom routine in MatLab. For both pulsation frequency and bulk lymph transport measurements, NIH ImageJ software was used for analysis.

To determine pulsation frequency, a region of interest (ROI) was drawn over a section of vessel that clearly showed lymph movement through the field-of-view. Changes in intensity as lymph flowed in/out through ROI were measured over entire stack using TimeSeriesAnalyzer (plugin available through NIH ImageJ). Mean intensity values produced were exported and plotted in Excel over time. Resulting pulsation traces are quantified by counting the number of peaks present over the 5 minute imaging sequence.

To determine bulk loading rate of dye, an ROI was drawn directly over the inguinal node. Changes in intensity as lymph loaded the node were measured over entire image stack using TimeSeriesAnalyzer (plugin available through NIH ImageJ). Mean intensity values produced were exported and plotted in Excel over time. Time to peak, max loading rate and dF/F were calculated from mean intensity values.

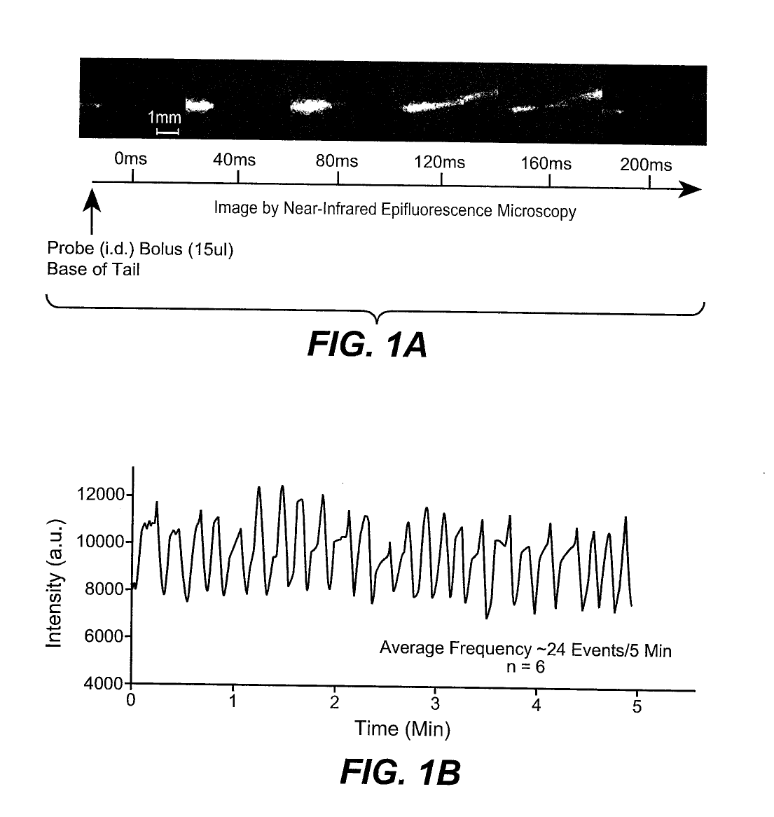

FIG. 1 illustrates the results from a lymph function assay measuring lymph pulsation frequency. FIG. 1A illustrates a representative time course images of pulsatile lymph movement through a vessel following injection of a 15 μl bolus of dye. FIG. 1B illustrates a baseline activity of −24 events/5 min, n=6 animals.

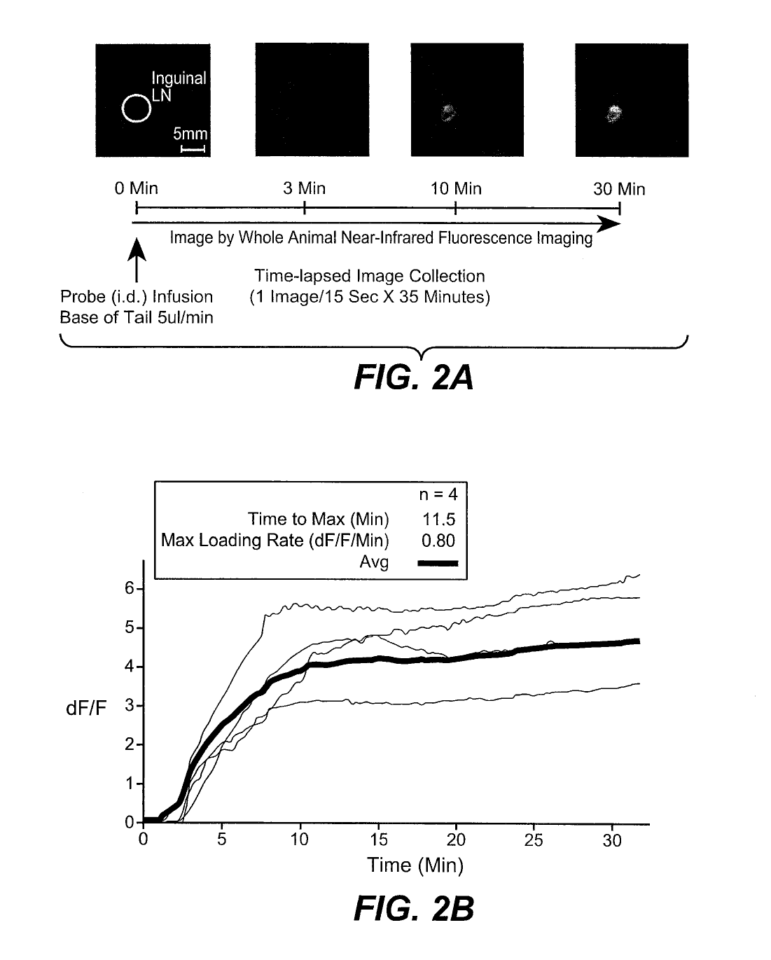

FIG. 2 illustrates the results from a lymph function assay measuring bulk lymph transport to an inguinal lymph node following infusion of 5 μL/min, 15 min dye near base of the tail at the start of imaging. FIG. 2A illustrates representative time course images show initial loading of inguinal node followed by axial node. FIG. 2B illustrates baseline loading rate and time to maximum signal intensity of inguinal node, n=4 animals.

### Example 2

### Measurement of Lymphatic Function to Monitor Efficacy of Anti-Cancer Agent

This example demonstrates monitoring the efficacy of anti-cancer agents (e.g, anti-VEGF-C or anti-NRP2) by measuring lymphatic function (e.g., lymph pulsation frequency and bulk lymph transport).

Female Balb-c nude mice were randomized into treatment groups as follows.

1) Control—No tumor

2) Control—Tumor

3) Anti-VEGFC 40 mg/kg, IP, 100 μl, 1×/week

4) Anti-VEGF 10 mg/kg IP, 100 μl, 1×/week

5) Anti-NRP2 40 mg/kg, IP, 100 μl, 1×/week

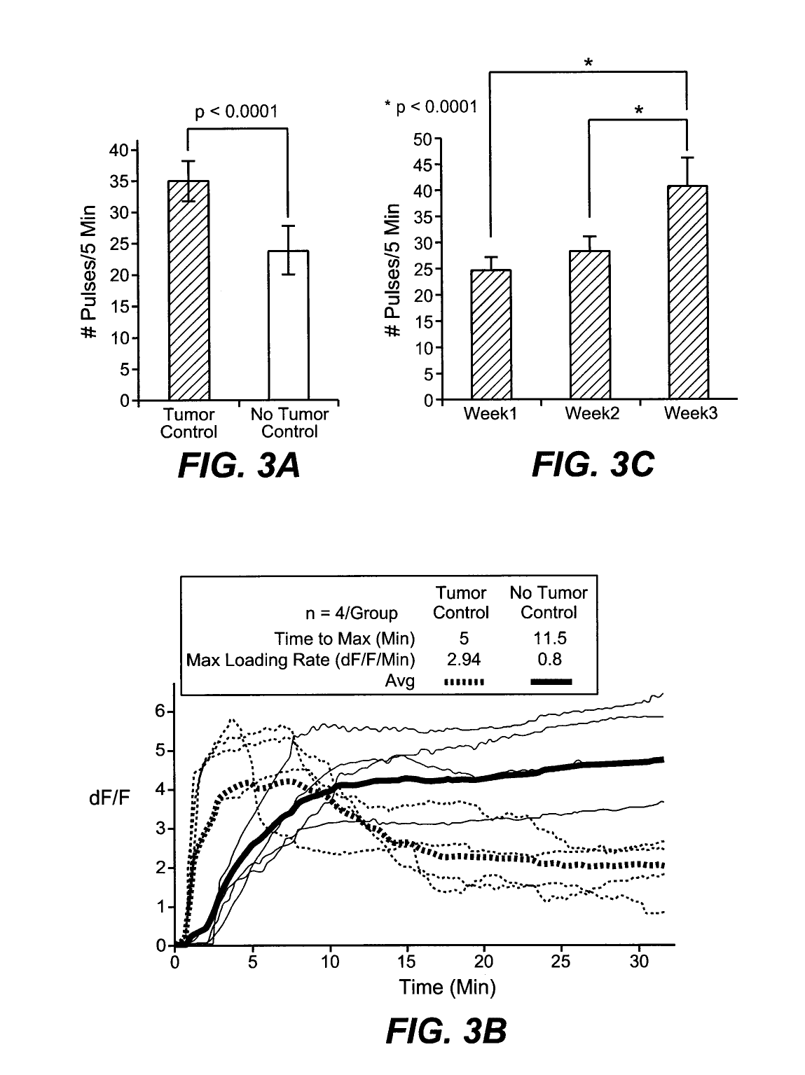

C6 rat glioblastoma cells (5.0×10⁵ cells in 200 μL PBS) were implanted subcutaneously into base of the tail of the mice, the right ear of the mice, or in the middle of the back approximately 20 mm above base of tail injection site. Mice were then not treated or treated with anti-VEGF-C(10 mg/kg), anti-VEGF-A(10 mg/kg), or anti-Nrp2(10 mg/kg) i.p. once a week for 3 weeks post implantation. Mice were sorted to give near identical mean tumor sizes before imaging and imaged at day 7, 14, and/or 21 post implantation. Mice were imaged as described in Example 1 above and the data is illustrated in FIGS. 3, 4, 5, 6, and 7.

FIG. 3 illustrates the results from a lymph function assay in mice, demonstrating that bulk lymph transport is up-regulated in tumor associated lymph networks. FIG. 3A illustrates data demonstrating that lymph pulsation frequency is up-regulated ˜%50 in tumor implanted mice, n=6 animals/group. FIG. 3B illustrates data demonstrating that bulk lymph transport is also up-regulated in tumor implanted mice, n=4 animals/group. FIG. 3C illustrates data demonstrating the time course of lymph pulsation up-regulation in tumor implanted mice, n=12 animals/group. Mice were sacrificed after 21 days due to tumor size.

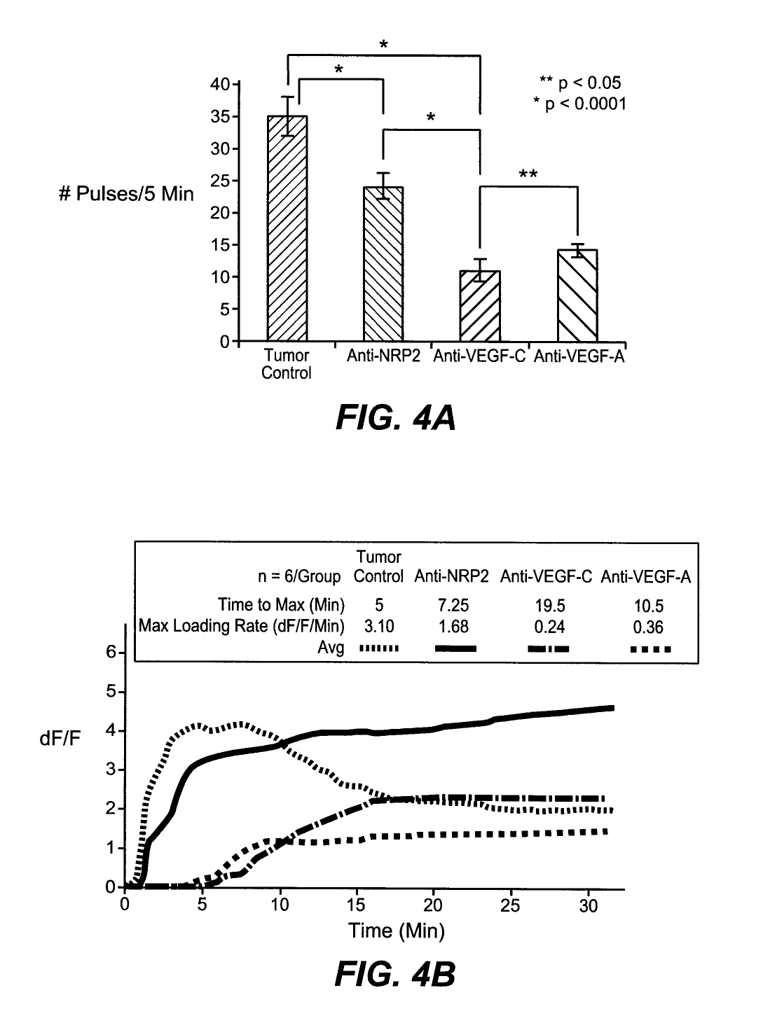

FIG. 4 illustrates data demonstrating that inhibition of the VEGF-C pathway decreases lymph transport in tumor associated networks. FIG. 4A illustrates data demonstrating that chronic treatment with anti-NRP2, anti-VEGF-C, or anti-VEGF-A in tumor-bearing mice significantly reduced lymph pulsation frequency, n=6 animals/group. FIG. 4B illustrates data demonstrating that chronic treatment with anti-NRP2, anti-VEGF-C, or anti-VEGF-A in tumor-bearing mice significantly reduced bulk lymph transport, n=6 animals/group.

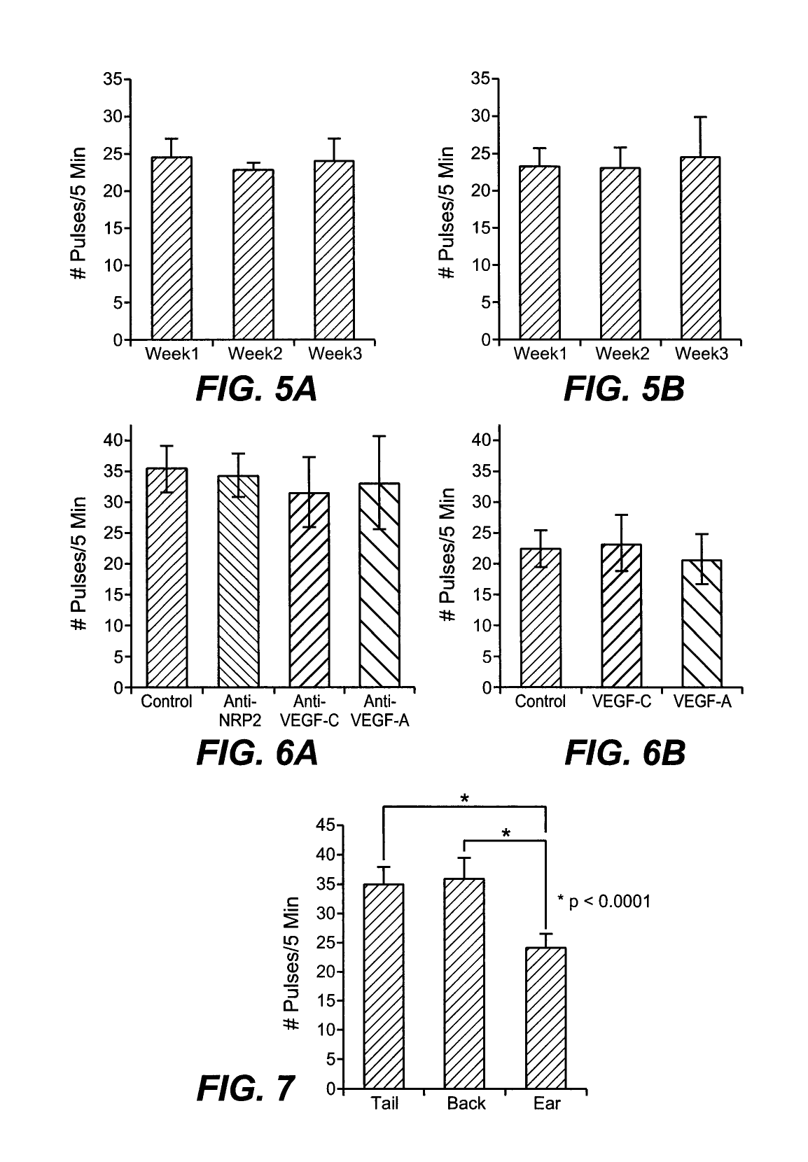

FIG. 5 illustrates data demonstrating that inhibition of the VEGF-C pathway did not significantly alter lymphatic function in non-tumor bearing mice. FIG. 5A illustrates data demonstrating that chronic treatment with anti-VEGF-C in non-tumor-bearing mice did not significantly change lymph pulsation frequency when measured over 3 weeks, n=6 animals/group. FIG. 5B illustrates data demonstrating that chronic treatment with anti-NRP2 in non-tumor-bearing mice did not significantly change lymph pulsation frequency when measured over 3 weeks, n=4 animals/group.

FIG. 6 illustrates data demonstrating that acute injection of anti-cancer agents does not change lymphatic function. FIG. 6A illustrates data demonstrating that acute injection of anti-NRP2, anti-VEGF-C, or anti-VEGF-A in tumor bearing mice does not result in any significant change in lymph pulsation frequency, n=6 animals/group. FIG. 6B illustrates data demonstrating that acute injection of recombinant VEGF-C protein or recombinant VEGF-A protein in non-tumor bearing mice does not result in any significant change in lymph pulsation frequency, n=6 animals/group.

FIG. 7 illustrates data demonstrating the specificity of up-regulation of lymphatic pulsation frequency. The data demonstrates that lymph pulsation frequency is up-regulated in both tail and back tumor bearing mice but is not up-regulated in the ear tumor bearing mice.

### Example 3

### Measurement of Lymph Pulsation to Determine Dose

This example describes measurement of lymph pulsation to determine the dose of a therapeutic agent (e.g., an anti-cancer agent), including, e.g., the minimal efficacious dose (MiED) or maximal efficacious dose (MxED) pre-clinically. Tumor bearing mice are treated with a placebo and a potential therapeutic agent at broad range of doses (for example 1 mg/kg to 200 mg/kg−1, 5, 10, 20, 40, 80, 150, 200 mg/kg) for 3 weeks. An imaging agent is administered to the mice so that the agent reaches the lymph vessels associated with the tumor draining lymph. Pulsation frequency is measured. The lowest dose where a statistically significant change in pulsation frequency compared to the pulsation frequency in the placebo treated group is defined as the MiED. The lowest dose where the maximal change in pulsation frequency (i.e. the doses where further increase provides no added changes in pulsation frequency) compared to pulsation frequency in the placebo treated group is defined as the MxED.

Pharmacokinetic analysis of the potential therapeutic agent at the MiED and MxED is performed using methods known in the art including those described in, e.g., Bagri et al., Clin Cancer Res. 16(15):3887-900 (2010)) to determine exposure defined by area under the curve (AUC), Cmax and Ctrough at both of these doses. The exposures determined at the MiED is the minimal exposure, and the exposures determined at the MxED are the target exposures.

During clinical studies in patients receiving the therapeutic agent, lymphatic pulsation rate is measured as described herein. Measurements are made prior to initial administration of the therapeutic agent and at a suitable time (e.g., 1, 2, 3, or more days or weeks) after each dose of the therapeutic agent. The mean pulsation rate and the mean change in pulsation rate is determined. The lowest dose where a statistically significant change in “on and post treatment” pulsation frequency compared to the pre-treatment frequency in the same patient is defined as the MiED. The lowest dose where the maximal change in pulsation frequency (i.e., the doses where further increase provides no added changes in pulsation frequency) “on and post treatment” compared to pre-treatment pulsation frequency is defined as the MxED.

Additionally, pharmacokinetic analysis of the agent is performed and the mean AUC, Cmax and Ctrough is determined for each dose group. Mean change in pulsation rate will be evaluated at exposures that are comparable to the minimal and target exposures determined in pre-clinical experiments to validate the pre-clinical analysis.

Although the foregoing invention has been described in some detail by way of illustration and example for purposes of clarity of understanding, the descriptions and examples should not be construed as limiting the scope of the invention. The disclosures of all patents, patent applications, scientific references, and Genbank Accession Nos. cited herein are expressly incorporated by reference in their entirety for all purposes as if each patent, patent application, scientific reference, and Genbank Accession No. were specifically and individually incorporated by reference.

What is claimed is:

1. A method of identifying a patient likely to be responsive to an anti-cancer agent, the method comprising:
(a) administering an imaging agent to a patient who has received at least one dose of an anti-cancer agent;
(b) detecting lymph pulsation frequency in a lymph vessel associated with a tumor draining lymph node in the patient; and
(c) comparing the lymph pulsation frequency to the pulsation frequency in the lymph vessel prior to treatment with the anti-cancer agent,
wherein a decrease in lymph pulsation frequency in the lymph vessel of at least about 10% identifies a patient who has an increased likelihood of being responsive to an anti-cancer agent.

2. The method of claim 1, wherein the lymph vessel connects the inguinal lymph node to the axial lymph node.

3. The method of claim 1, wherein the imaging agent comprises a fluorescent dye.

4. The method of claim 3, wherein the fluorescent dye is Alexafluor680.

5. The method of claim 3, wherein lymph pulsation frequency is detected using fluorescence microscopy.

6. The method of claim 1, wherein the patient is a human.

7. The method of claim 1, wherein the patient has been diagnosed with a cancer selected from the group consisting of: colorectal cancer, breast cancer, lung cancer, glioblastoma, renal cancer, and combinations thereof.

8. The method of claim 1 further comprising
(d) administering an effective amount of an anti-cancer agent to the patient if a decrease in lymph pulsation frequency in the lymph vessel of at least about 10% is detected.

9. The method of claim 8, wherein the anti-cancer agent is a member selected from the group consisting of: an NRP2 antagonist, a VEGF-C antagonist, and combinations thereof.

10. The method of claim 9, wherein the NRP2 antagonist is an anti-NRP2 antibody.

11. The method of claim 9, wherein the VEGF-C antagonist is an anti-VEGF-C antibody.

12. The method of claim 8 further comprising
(e) administering an effective amount of a second anti-cancer agent to the patient.

13. The method of claim 12, wherein the second anti-cancer agent is a VEGF antagonist.

14. The method of claim 13, wherein the VEGF antagonist is an anti-VEGF antibody.

15. The method of claim 14, wherein the anti-VEGF antibody is bevacizumab.

16. A method of identifying a patient who has an increased likelihood of undergoing metastasis, the method comprising:
(a) administering an imaging agent to a patient who has received at least one dose of an anti-cancer agent;
(b) detecting lymph pulsation frequency in a lymph vessel associated with a tumor draining lymph node in the patient; and
(c) comparing the lymph pulsation frequency to the pulsation frequency in the lymph vessel prior to treatment with the anti-cancer agent,
wherein an increase in the lymph pulsation frequency in the lymph vessel of at least about 10% identifies a patient who has an increased likelihood of undergoing metastasis.

17. The method of claim 16, wherein the lymph vessel connects the inguinal lymph node to the axial lymph node.

18. The method of claim 16, wherein the imaging agent comprises a fluorescent dye.

19. The method of claim 18, wherein the fluorescent dye is Alexafluor680.

20. The method of claim 18, wherein lymph pulsation frequency is detected using fluorescence microscopy.

21. The method of claim 16, wherein the patient is a human.

22. The method of claim 16, wherein the patient has been diagnosed with a cancer selected from the group consisting of: colorectal cancer, breast cancer, lung cancer, glioblastoma, renal cancer, and combinations thereof.

23. The method of claim 1 further comprising
(d) administering an effective amount of an anti-cancer agent to the patient if an increase in lymph pulsation frequency in the lymph vessel of at least about 10% is detected.

24. The method of claim 23, wherein the anti-cancer agent is a member selected from the group consisting of: an NRP2 antagonist, a VEGF-C antagonist, and combinations thereof.

25. The method of claim 24, wherein the NRP2 antagonist is an anti-NRP2 antibody.

26. The method of claim 24, wherein the VEGF-C antagonist is an anti-VEGF-C antibody.

27. The method of claim 23 further comprising
(e) administering an effective amount of a second anti-cancer agent to the patient.

28. The method of claim 27, wherein the second anti-cancer agent is a VEGF antagonist.

29. The method of claim 28, wherein the VEGF antagonist is an anti-VEGF antibody.

30. The method of claim 29, wherein the anti-VEGF antibody is bevacizumab.

31. A method of for monitoring the effectiveness of anti-cancer therapy, the method comprising:
(a) administering an imaging agent to a patient who has received at least one dose of an anti-cancer agent;
(b) detecting lymph pulsation frequency in a lymph vessel associated with a tumor draining lymph node in the patient; and
(c) comparing the lymph pulsation frequency to the pulsation frequency in the lymph vessel prior to treatment with the anti-cancer agent,
wherein a decrease in lymph pulsation frequency in the lymph vessel of at least about 10% identifies an effective anticancer agent.

32. The method of claim 31, wherein the lymph vessel connects the inguinal lymph node to the axial lymph node.

33. The method of claim 31, wherein the imaging agent comprises a fluorescent dye.

34. The method of claim 33, wherein the fluorescent dye is Alexafluor680.

35. The method of claim 33, wherein lymph pulsation frequency is detected using fluorescence microscopy.

36. The method of claim 31, wherein the patient is a human.

37. The method of claim 31, wherein the patient has been diagnosed with a cancer selected from the group consisting of: colorectal cancer, breast cancer, lung cancer, glioblastoma, renal cancer, and combinations thereof.

38. The method of claim 31 further comprising
(d) administering an effective amount of an anti-cancer agent to the patient if a decrease in lymph pulsation frequency in the lymph vessel of at least about 10% is detected.

39. The method of claim 38, wherein the anti-cancer agent is a member selected from the group consisting of: an NRP2 antagonist, a VEGF-C antagonist, and combinations thereof.

40. The method of claim 39, wherein the NRP2 antagonist is an anti-NRP2 antibody.

41. The method of claim 39, wherein the VEGF-C antagonist is an anti-VEGF-C antibody.

42. The method of claim 38 further comprising
(e) administering an effective amount of a second anti-cancer agent to the patient.

43. The method of claim 42, wherein the second anti-cancer agent is a VEGF antagonist.

44. The method of claim 43, wherein the VEGF antagonist is an anti-VEGF antibody.

45. The method of claim 44, wherein the anti-VEGF antibody is bevacizumab.

46. A method of optimizing dose of an anti-cancer agent, the method comprising:
(a) administering an imaging agent to a patient who has received at least one dose of an anti-cancer agent;
(b) detecting lymph pulsation frequency in a lymph vessel associated with a tumor draining lymph node in the patient; and
(c) comparing the lymph pulsation frequency to the pulsation frequency in the lymph vessel prior to treatment with the anti-cancer agent,
wherein a change in lymph pulsation frequency in the lymph vessel identifies the dose as an effective dose.

47. The method of claim 46, wherein the anti-cancer agent is a member selected from the group consisting of: an NRP2 antagonist, a VEGF-C antagonist, and combinations thereof.

48. A method of optimizing dose of an anti-cancer agent, the method comprising:
(a) administering an imaging agent to a patient who has received at least one dose of an anti-cancer agent;
(b) detecting lymph pulsation frequency in a lymph vessel associated with a tumor draining lymph node in the patient; and
(c) comparing the lymph pulsation frequency to the pulsation frequency in the lymph vessel prior to treatment with the anti-cancer agent,
wherein no change in lymph pulsation frequency identifies the dose as a maximum effective dose.

49. The method of claim 48, wherein the anti-cancer agent is a member selected from the group consisting of: an NRP2 antagonist, a VEGF-C antagonist, and combinations thereof.
